(19) United States
(12) Patent Application Publication
(10) Pub. No.: US 2016/0220133 A1
(43) Pub. Date: Aug. 4, 2016

(54) BIOLOGICAL INFORMATION MEASURING APPARATUS

(71) Applicant: Seiko Epson Corporation, Tokyo (JP)

(72) Inventor: Akira Inagaki, Matsumoto-shi (JP)

(21) Appl. No.: 15/005,858

(22) Filed: Jan. 25, 2016

(30) Foreign Application Priority Data
Feb. 4, 2015 (JP) 2015-020012

(51) Int. Cl. A61B 5/024; A61B 5/00

(52) U.S. Cl. CPC A61B 5/02427; A61B 5/742; A61B 5/681; A61B 2562/0219; A61B 2560/0209

(57) ABSTRACT

A biological information measuring apparatus includes a light emitting unit that emits light to a test subject, a light receiving unit that receives light which is reflected from the test subject, and a controller that determines whether at least one of the light receiving unit and the light emitting unit is brought into an intermittent operation.

### CROSS-REFERENCES TO RELATED APPLICATIONS

This application claims priority to Japanese Patent Application No. 2015-020012, filed Feb. 4, 2015, the entirety of which is herein incorporated by reference.

### BACKGROUND

1. Technical Field

The present invention relates to a biological information measuring apparatus that measures pulse waves and the like.

2. Related Art

Hitherto, there have been known measuring apparatuses that are worn around body parts, such as a wrist, by a band or the like and measure biological information such as wearer's pulse waves using an optical or ultrasonic pulse wave detection sensor, and wristwatch type electronic apparatuses having a function of measuring the biological information. For example, WO 2014/091424 discloses a biological information measuring apparatus which is worn around the arm (wrist) of a test subject (user) and measures biological information such as pulse waves using an optical pulse wave sensor. Such apparatuses (measuring apparatus, electronic apparatus) optically measure the flow of blood under a skin surface by emitting light toward a test subject and receiving light reflected from the test subject and convert the measured blood flow into a signal to thereby obtain biological information such as pulse waves.

In such a biological information measuring apparatus, an operable time is required to be increased by power saving. However, WO 2014/091424 does not disclose power saving, and does not disclose control of the execution of an intermittent operation for power saving or control of a stop time and a measurement time of an intermittent operation.

### SUMMARY

An advantage of some aspects of the invention is to solve at least a part of the problems described above, and the invention can be implemented as the following forms or application examples.

### Application Example 1

A biological information measuring apparatus according to this application example includes a light emitting unit that emits light to a test subject, a light receiving unit that receives light which is reflected from the test subject, and a controller that determines whether at least one of the light receiving unit and the light emitting unit is brought into an intermittent operation.

According to this application example, the controller of the biological information measuring apparatus determines whether an intermittent operation is performed in accordance with conditions of the use of the biological information measuring apparatus, measured biological information, or the like. The intermittent operation is set, and thus the power saving of the biological information measuring apparatus is achieved. Therefore, it is possible to provide the biological information measuring apparatus of which the operable time is automatically increased.

### Application Example 2

It is preferable that the biological information measuring apparatus according to the application example further includes a display unit that gives notice of information, and that the controller notifies the display unit that switching from a successive operation to the intermittent operation has been performed.

According to this application example, the biological information measuring apparatus includes the display unit, and the controller notifies the display unit of information indicating that switching from a successive operation to an intermittent operation has been performed. Thereby, it is possible to make a test subject (user) recognize that switching from a successive operation to an intermittent operation has been performed.

### Application Example 3

In the biological information measuring apparatus according to the application example, it is preferable that the controller sets at least one of a stop time and a measurement time of the intermittent operation.

According to this application example, the controller of the biological information measuring apparatus sets at least one of a stop time and a measurement time of an intermittent operation in accordance with information which is input by a user, conditions of the use of the biological information measuring apparatus, measured biological information, or the like. Thereby, the biological information measuring apparatus performs an intermittent operation, and thus power saving is achieved. Therefore, it is possible to provide the biological information measuring apparatus of which the operable time is automatically increased.

### Application Example 4

It is preferable that the biological information measuring apparatus according to the application example further includes a secondary battery that accumulates power, and that the controller sets at least one of the stop time and the measurement time on the basis of at least one of a charging amount, remaining amount, and consumption of the secondary battery.

According to this application example, the controller of the biological information measuring apparatus sets at least one of a stop time and a measurement time of an intermittent operation in accordance with at least one of a charging amount, remaining amount, and consumption of the secondary battery. Thereby, the biological information measuring apparatus performs an intermittent operation, and thus power saving is achieved. In the biological information measuring apparatus, an operable time is automatically increased, and thus measurement results (history) can remain continuously.

### Application Example 5

In the biological information measuring apparatus according to the application example, it is preferable that the controller sets at least one of the stop time and the measurement time on the basis of a use time which is input by the test subject.

According to this application example, a user inputs a time when measurement is desired to be performed, and thus the controller of the biological information measuring apparatus sets at least one of a stop time and a measurement time of an intermittent operation. Thereby, the biological information measuring apparatus performs an intermittent operation, and thus power saving is achieved. In the biological information measuring apparatus, an operable time is increased in accordance with a use time which is input, and thus measurement according to user's situation can be performed.

### Application Example 6

In the biological information measuring apparatus according to the application example, it is preferable that the controller sets at least one of the stop time and the measurement time on the basis of biological information on the test subject which was measured in the past.

According to this application example, the controller of the biological information measuring apparatus sets at least one of a stop time and a measurement time of an intermittent operation according to a user's health condition with reference to biological information which was measured in the past. Thereby, the biological information measuring apparatus performs an intermittent operation, and thus power saving is achieved. Since the biological information measuring apparatus automatically performs an intermittent operation according to a user's health condition, an operable time is increased, and thus measurement results (history) of necessary biological information measurement results (history) can remain continuously.

### Application Example 7

It is preferable that the biological information measuring apparatus according to the application example further includes a plurality of the light emitting units.

According to this application example, the biological information measuring apparatus includes the plurality of light emitting units in order to improve measurement accuracy. An intermittent operation is applied to such a biological information measuring apparatus, and thus it is possible to increase an operable time of the biological information measuring apparatus capable of measuring biological information with a high level of accuracy.

### Application Example 8

In the biological information measuring apparatus according to the application example, in a state of being set to the intermittent operation, it is preferable that the controller notifies the display unit of contents of the set intermittent operation.

According to this application example, the controller of the biological information measuring apparatus displays contents of a stop time and a measurement time, which are set in order to perform an intermittent operation, on the display unit. Thereby, a user can always ascertain the biological information measuring apparatus being in an intermittent operation mode and the contents of the intermittent operation and can determine whether the set contents are appropriate in the present action.

### Application Example 9

In the biological information measuring apparatus according to the application example, in a state of being set to the intermittent operation, it is preferable that the controller notifies the display unit of being in the measurement time.

According to this application example, the controller of the biological information measuring apparatus performs a display indicating that biological information is actually measured, on the display unit. Thereby, a user can recognize the time when the biological information measuring apparatus is performing measurement during an intermittent operation. In addition, the user himself or herself can determine whether contents of the set intermittent operation are appropriate.

### Application Example 10

In the biological information measuring apparatus according to the application example, it is preferable that the controller notifies the display unit of an operable time.

According to this application example, the controller of the biological information measuring apparatus always displays an operable time on the display unit, and thus a user can prevent the continuous recording of biological information from being stopped due to forgetfulness of charging a secondary battery, or the like.

### Application Example 11

It is preferable that the biological information measuring apparatus according to the application example further includes an acceleration sensor.

According to this application example, the biological information measuring apparatus includes the acceleration sensor, and thus it is possible to detect the state (sleep, stress, active mass, or the like) of a user from, for example, pulse waves and measurement results of the acceleration sensor.

### Application Example 12

In the biological information measuring apparatus according to the application example, it is preferable that the controller sets an intermittent operation when the test subject is in a sleep state.

According to this application example, the active mass of a human being is decreased in a sleep state, and thus a fluctuation in pulse waves is reduced. For this reason, the controller of the biological information measuring apparatus sets an intermittent operation when detecting a user's sleep state. Thereby, an operable time of the biological information measuring apparatus is increased.

### Application Example 13

In the biological information measuring apparatus according to the application example, it is preferable that the controller sets an intermittent operation on the basis of the test subject's lifestyle which is estimated by biological information of the test subject which was measured in the past.

According to this application example, the controller of the biological information measuring apparatus sets an intermittent operation on the basis of a user's lifestyle. Thereby, an operable time of the biological information measuring apparatus is increased.

### BRIEF DESCRIPTION OF THE DRAWINGS

The invention will be described with reference to the accompanying drawings, wherein like numbers reference like elements.

FIG. 1A is a schematic diagram illustrating the exterior of a biological information measuring apparatus according to a first embodiment when the biological information measuring apparatus is seen from the front side, and FIG. 1B is a diagram illustrating the exterior of the biological information measuring apparatus when the biological information measuring apparatus of FIG. 1A is obliquely seen from above.

FIG. 2 is a diagram illustrating the exterior of the biological information measuring apparatus of FIG. 1A when the biological information measuring apparatus is seen from the side.

FIG. 3 is a schematic diagram illustrating the wearing of a biological information measuring apparatus and communication with a terminal device.

FIG. 4 is a functional block diagram of a biological information measuring apparatus.

FIG. 5 is a front cross-sectional view illustrating a detailed configuration example of a sensor unit.

FIG. 6 is a flow chart illustrating setting of whether a biological information measuring apparatus is brought into an intermittent operation.

FIG. 7A is a diagram illustrating a notification screen during a measurement time in an intermittent operation, and FIG. 7B is a diagram illustrating a notification screen during a stop time in an intermittent operation.

FIG. 8 is a flow chart illustrating an intermittent operation of a biological information measuring apparatus according to a second embodiment.

FIG. 9 is a diagram illustrating an example of biological information measured in the past.

FIG. 10 is a flow chart illustrating an intermittent operation of a biological information measuring apparatus according to a third embodiment.

FIG. 11 is a flow chart illustrating an intermittent operation of a biological information measuring apparatus according to a fourth embodiment.

FIG. 12 is a flow chart illustrating an intermittent operation of a biological information measuring apparatus according to a fifth embodiment.

FIG. 13 is a flowchart illustrating an intermittent operation of a biological information measuring apparatus according to a sixth embodiment.

FIG. 14 is a flowchart illustrating an intermittent operation of a biological information measuring apparatus according to a seventh embodiment.

FIG. 15 is a table illustrating stop times according to exercise types.

FIG. 16 is a cross-sectional view illustrating a heart rate monitoring apparatus as a biological information measuring apparatus which is an example of the related art.

FIG. 17 is a perspective view illustrating a heart rate monitoring apparatus as a biological information measuring apparatus according to an eighth embodiment.

FIG. 18 is a side view illustrating a heart rate monitoring apparatus as a biological information measuring apparatus according to a ninth embodiment.

FIG. 19 is a perspective view illustrating a heart rate monitoring apparatus as a biological information measuring apparatus according to a tenth embodiment.

FIG. 20 is a cross-sectional view illustrating a heart rate monitoring apparatus as a biological information measuring apparatus according to an eleventh embodiment.

FIG. 21 is a flow chart illustrating a method of manufacturing a biological information measuring apparatus.

FIG. 22 is a schematic diagram illustrating a web page serving as a starting point of a health manager in a biological information measuring apparatus according to a twelfth embodiment.

FIG. 23 is a diagram illustrating an example of a nutrition web page.

FIG. 24 is a diagram illustrating an example of an activity level web page.

FIG. 25 is a diagram illustrating an example of a mental concentration web page.

FIG. 26 is a diagram illustrating an example of a sleep web page.

FIG. 27 is a diagram illustrating an example of a daily activity web page.

FIG. 28 is a diagram illustrating an example of a health degree web page.

### DESCRIPTION OF EXEMPLARY EMBODIMENTS

Hereinafter, embodiments of the invention will be described with reference to the accompanying drawings. Meanwhile, in the following drawings, since each layer and each member have a recognizable size, the scale of each layer and each member is different from the actual scale.

### First Embodiment

### Overall Configuration Example of Biological Information Measuring Apparatus

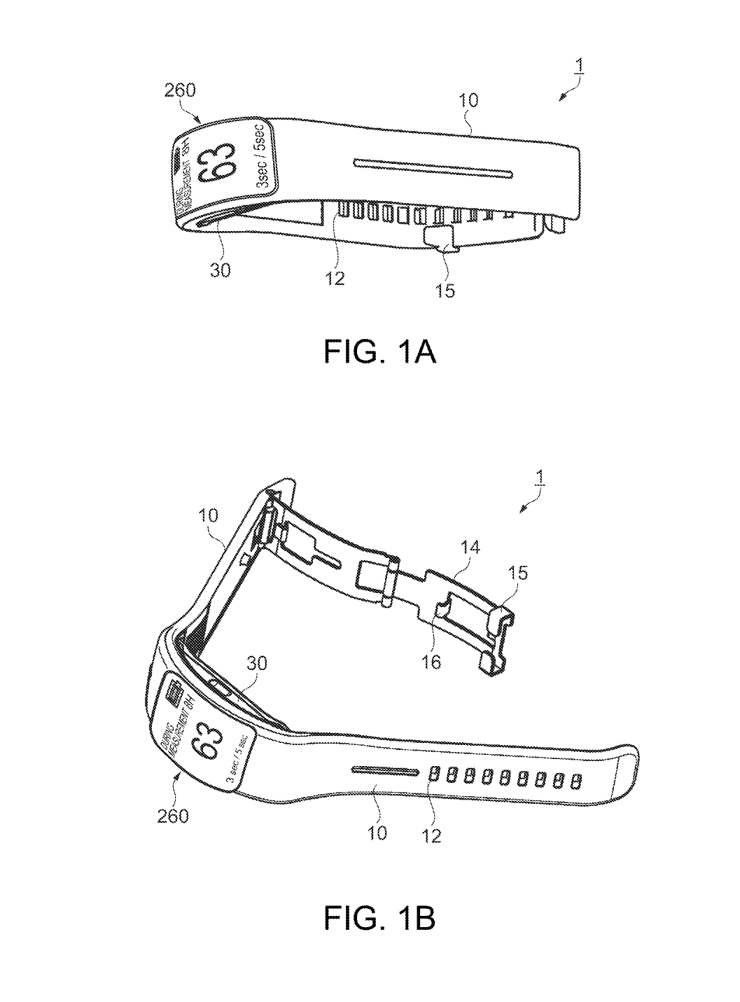

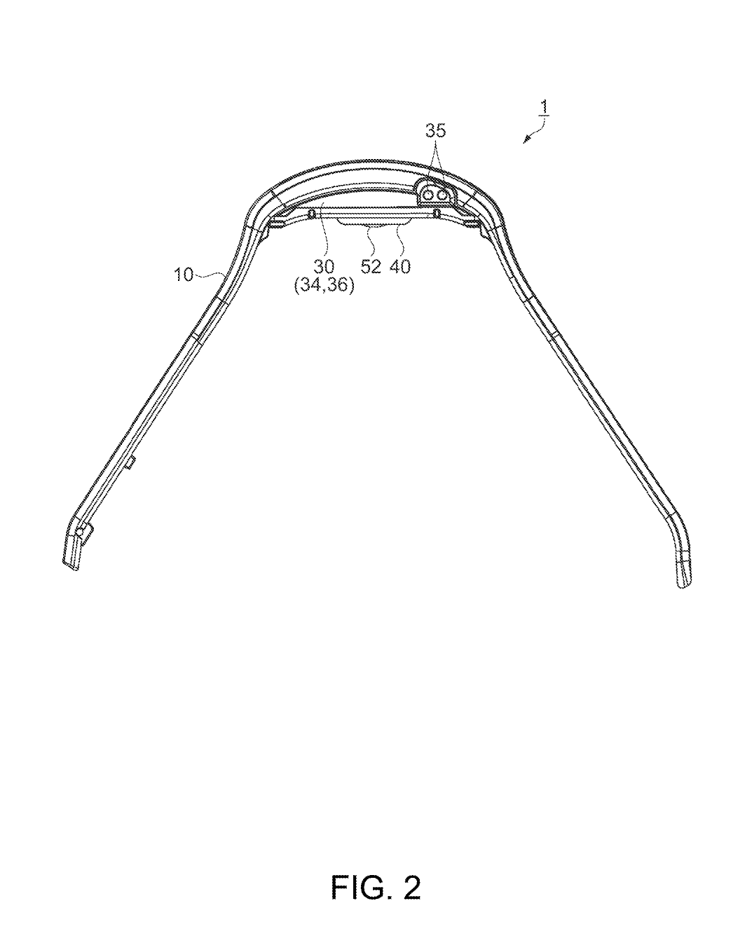

FIGS. 1A and 1B and FIG. 2 are schematic diagrams illustrating the exterior of a biological information measuring apparatus (biological information detecting apparatus) according to a first embodiment. FIG. 1A is a diagram when the biological information measuring apparatus is seen from the front, FIG. 1B is a diagram when the biological information measuring apparatus of FIG. 1A is obliquely seen from above, and FIG. 2 is a diagram when the biological information measuring apparatus is seen from the side. Meanwhile, the biological information measuring apparatus of this embodiment is not limited to the configurations illustrated in FIGS. 1A and 1B and FIG. 2, and various modifications such as the omission of some of the components thereof, replacement with other components, or the addition of other components can be made.

As illustrated in FIGS. 1A and 1B and FIG. 2, a biological information measuring apparatus 1 of this embodiment includes a band portion 10 and a case portion 30. The case portion 30 is attached to the band portion 10. The band portion 10 is wound around the wrist of a wearer (hereinafter, also referred to as a user) so that the biological information measuring apparatus 1 is worn thereon. The band portion 10 includes band holes 12 and a buckle portion 14. The buckle portion 14 includes a band insertion portion 15 and a protrusion portion 16. The user inserts one end side of the band portion 10 into the band insertion portion 15 of the buckle portion 14 and inserts the protrusion portion 16 of the buckle portion 14 into the band hole 12 of the band portion 10 to thereby wear the biological information measuring apparatus 1 around his or her wrist.

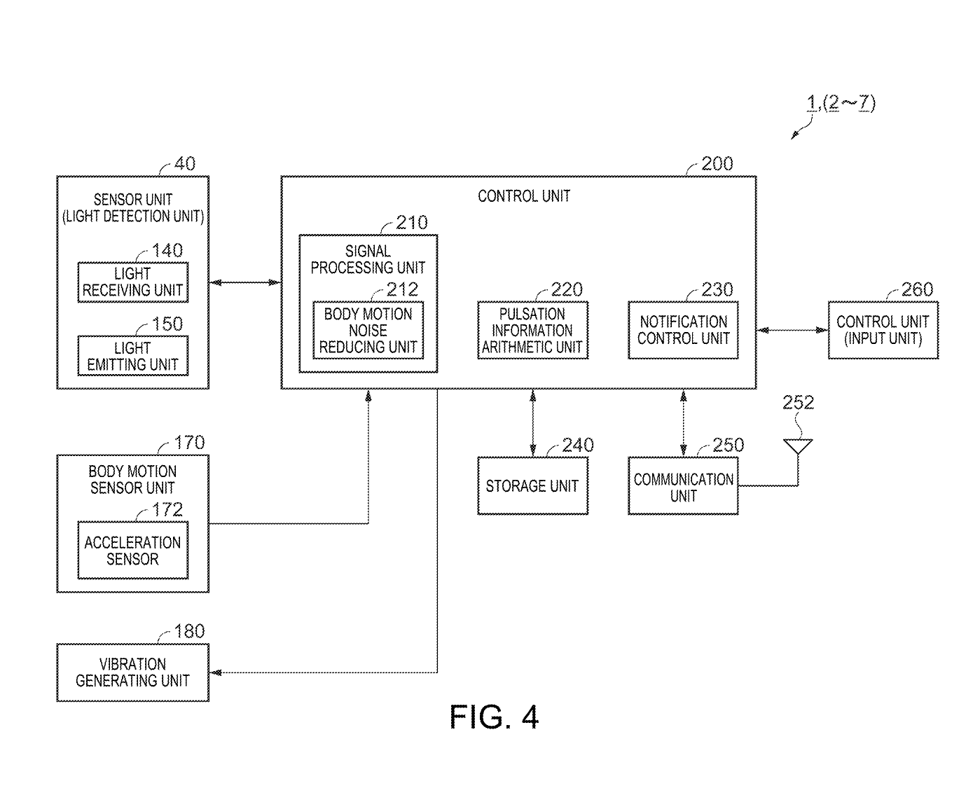

The case portion 30 is equivalent to a main body portion of the biological information measuring apparatus 1. Various components of the biological information measuring apparatus 1 such as the sensor unit 40 and a control unit 200 (see FIG. 4), and a secondary battery that accumulates power are provided within the case portion 30. That is, the case portion 30 is a housing that accommodates the components. The case portion 30 includes, for example, a top case 34 which is positioned on the opposite side to the wrist and a bottom case 36 which is positioned on the wrist side. A display unit 260 is provided on the top case side of the case portion 30. Meanwhile, the case portion 30 may not be configured so as to separate into the top case 34 and the bottom case 36.

The display unit 260 displays various pieces of information such as a time, measured biological information, and setting information for bringing the biological information measuring apparatus 1 into an intermittent operation, and is constituted by, for example, a liquid crystal display, an organic EL display, or an electronic paper display. In particular, when the display unit is constituted by an organic EL display or an electronic paper display, the display unit is driven while saving power, and thus measurement can be performed for a longer time.

In addition, the display unit 260 includes a touch-panel type input unit. Information displayed on the display unit 260 is selected and input by performing an input operation such as tapping. The touch panel is constituted by an electrostatic capacitive type touch panel, a resistive film type touch panel, or the like. The touch panel (display unit 260) is used as an input unit at the time of setting whether at least one of a light receiving unit 140 and a light emitting unit 150 to be described later is brought into an intermittent operation.

As illustrated in FIG. 2, the case portion 30 is provided with a terminal portion 35. When the biological information measuring apparatus 1 is mounted on a cradle not shown in the drawing, a terminal portion of the cradle and the terminal portion 35 of the case portion 30 are electrically connected to each other. Thereby, a secondary battery (battery) provided in the case portion 30 can be charged.

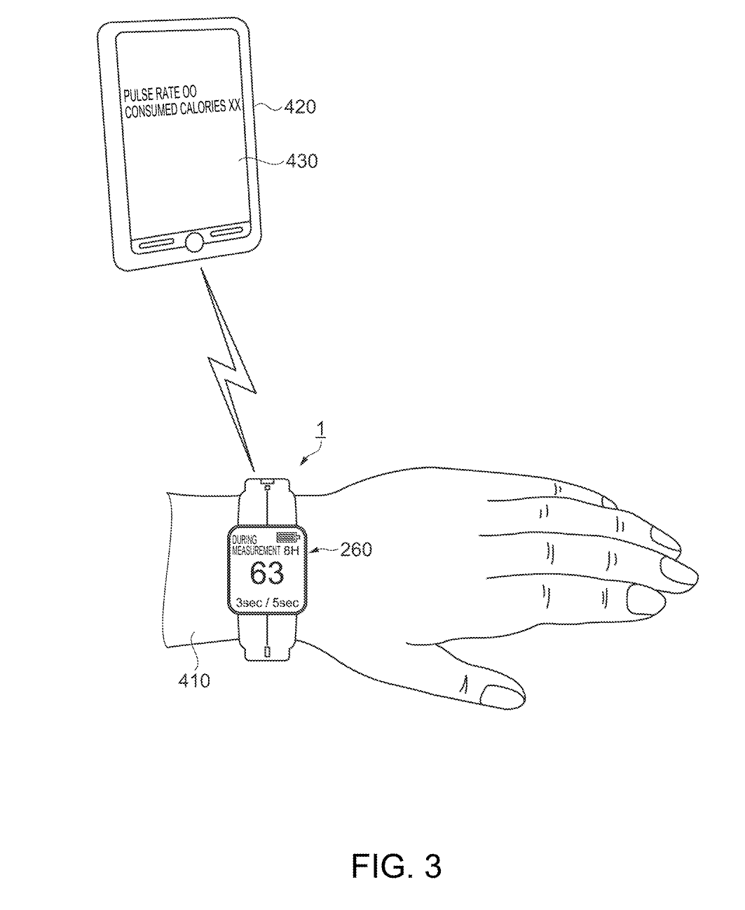

FIG. 3 is a schematic diagram illustrating the wearing of the biological information measuring apparatus 1 and communication with a terminal device 420. As illustrated in FIG. 3, a user who is a test subject wears the biological information measuring apparatus 1 around a wrist 410 like a timepiece. As illustrated in FIG. 2, a light emitting surface and a light receiving surface of the sensor unit 40 are provided on a surface of the case portion 30 on a test subject side. Accordingly, when the biological information measuring apparatus 1 is worn, a convex portion 52 of the sensor unit 40 comes into contact with the skin surface of the wrist 410 and applies pressure thereto, the light emitting unit 150 of the sensor unit 40 emits light in this state, and the light receiving unit 140 receives reflected light, and thus biological information such as pulse waves is detected.

The biological information measuring apparatus 1 and the terminal device 420 are connected to each other for communication, and thus data can be exchanged therebetween. The terminal device 420 is a portable communication terminal such as a smartphone or a feature phone. Alternatively, the terminal device 420 may be an information processing terminal such as a tablet computer. Short-distance wireless communication such as Bluetooth (registered trademark) can be adopted as a communication connection between the biological information measuring apparatus 1 and the terminal device 420. In addition, the connection may be performed by wired communication such as Ethernet (registered trademark).

A display area of the display unit 260 included in the wristwatch type biological information measuring apparatus 1 is narrow, and the amount of information capable of being displayed in the display area is limited. In this manner, the biological information measuring apparatus 1 and the terminal device 420 are connected to each other for communication, and thus detailed information such as a pulse rate and consumed calories, data obtained by graphing accumulated biological information, and the like can be displayed on the display unit 430 (LCD or the like) of the terminal device 420. In contrast, when an e-mail or the like is received in the terminal device 420, the information can be transported to the biological information measuring apparatus 1 from the terminal device 420. Meanwhile, the arithmetic processing of information such as a pulse rate or consumed calories may be performed by the biological information measuring apparatus 1, or at least a portion thereof may be performed by the terminal device 420.

FIG. 4 is a functional block diagram of the biological information measuring apparatus. As illustrated in FIG. 4, the biological information measuring apparatus 1 includes the sensor unit 40, a body motion sensor unit 170, a vibration generating unit 180, the control unit 200, a storage unit 240, a communication unit 250, an antenna 252, and the display unit 260.

The sensor unit 40 detects biological information such as pulse waves, and includes the light receiving unit 140 and the light emitting unit 150. A pulse wave sensor (photoelectric sensor) is realized by the light receiving unit 140, the light emitting unit 150, and the like. The sensor unit 40 outputs a signal detected by the pulse wave sensor as a pulse wave detection signal.

The body motion sensor unit 170 outputs a body motion detection signal which is a signal varying in response to body motion, on the basis of pieces of sensor information of various sensors. The body motion sensor unit 170 includes, for example, an acceleration sensor 172 as a body motion sensor. Meanwhile, the body motion sensor unit 170 may include a pressure sensor, a gyro sensor, or the like as the body motion sensor.

The control unit 200 performs various types of signal processes and control processes, for example, with the storage unit 240 as a work area, and can be realized by, for example, a processor such as a CPU or a logic circuit such as an ASIC. The control unit 200 includes a signal processing unit 210, a pulsation information arithmetic unit 220, and a notification control unit 230.

The signal processing unit 210 performs various types of signal processes (filtering and the like), and performs signal processing on, for example, a pulse wave detection signal from the sensor unit 40, a body motion detection signal from the body motion sensor unit 170, or the like. For example, the signal processing unit 210 includes a body motion noise reducing unit 212. The body motion noise reducing unit 212 performs processing for reducing (removing) body motion noise which is noise caused by body motion, from the pulse wave detection signal, on the basis of the body motion detection signal from the body motion sensor unit 170. Specifically, the body motion noise reducing unit performs a noise reduction process using an adaptive filter or the like.

The pulsation information arithmetic unit 220 performs arithmetic processing of pulsation information on the basis of a signal from the signal processing unit 210, and the like. The pulsation information is information such as, for example, a pulse rate. Specifically, the pulsation information arithmetic unit 220 obtains a spectrum by performing frequency analysis processing such as fast Fourier transform (FFT) on the pulse wave detection signal having been subjected to the noise reduction process by the body motion noise reducing unit 212, and performs a process of setting a representative frequency in the obtained spectrum as a frequency of a heartbeat. A value obtained by increasing the obtained frequency by 60 times is set to be a pulse rate (heart rate) which is generally used. Meanwhile, the pulsation information is not limited to the pulse rate itself, and may be various other pieces of information (for example, the frequency or cycle of a heartbeat) which indicate, for example, a pulse rate. In addition, the pulsation information may be information indicating the state of pulsation, or a value indicating, for example, the amount of blood itself may be set as pulsation information.

The notification control unit 230 controls the display unit 260. The display unit 260 displays various pieces of information to a user under the control of the notification control unit 230. In addition, the notification control unit 230 controls the vibration generating unit 180. The vibration generating unit 180 notifies a user of various pieces of information by vibration. The vibration generating unit 180 can be realized by, for example, a vibration motor (vibrator). The vibration motor generates vibration, for example, by rotating an eccentric weight. Specifically, the eccentric weight is attached to both ends of a driving shaft (rotor shaft) so that the motor itself shakes. The vibration of the vibration generating unit 180 is controlled by the notification control unit 230. Meanwhile, the vibration generating unit 180 is not limited to such a vibration motor, and various modifications can be made. The vibration generating unit 180 may be realized by, for example, a piezo element.

For example, a notice of start-up at the time of power-on, a notice of the first success in detecting pulse waves, a warning when a pulse-wave undetectable state is continued for a fixed period of time, a notice at the time of the movement of a fat combustion zone, a warning at the time of a battery voltage drop, a notice of a wake-up alarm, or a notice of an e-mail or a call from a terminal device such as a smartphone can be performed by the vibration of the vibration generating unit 180. Meanwhile, the pieces of information may be displayed on the display unit 260, or may use both the vibration generating unit 180 and the display unit 260.

The communication unit 250 performs communication with the external terminal device 420 as described in FIG. 3. For example, the communication unit performs wireless communication according to a standard such as Bluetooth (registered trademark). Specifically, the communication unit 250 receives a signal from the antenna 252 and transmits a signal to the antenna 252. The function of the communication unit 250 can be realized by a processor for communication or a logic circuit such as an ASIC.

The storage unit 240 stores measurement results of biological information, various types of tables, or the like, secures programs of the control unit 200, a work area, and the like, and includes a storage element such as a random access memory (RAM) or an electrically erasable programmable read-only memory (EEPROM).

### Configuration Example of Sensor Unit

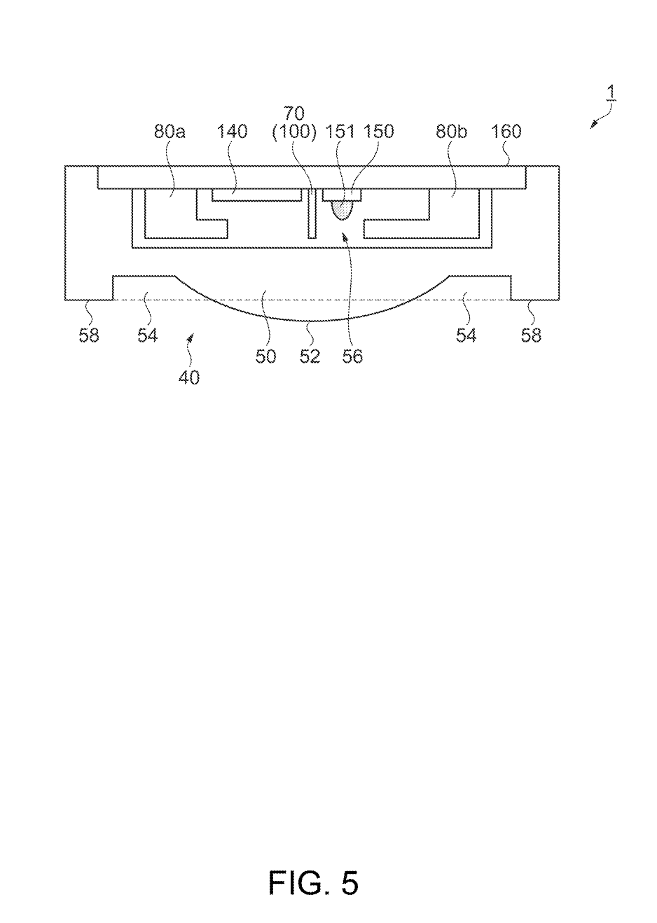

Next, a configuration example of the sensor unit 40 will be described. FIG. 5 is a front cross-sectional view illustrating a detailed configuration example of the sensor unit 40.

As illustrated in FIG. 5, the sensor unit 40 is constituted by a light detection unit including a substrate 160, the light emitting unit 150, the light receiving unit 140, a light shielding member 70, and a throttle portion 80 (80a and 80b), and other members. The other members include the convex portion 52, a groove portion 54, a concave portion 56, a pressing suppressing portion 58, and the like which are realized by a light transmitting member 50.

The light receiving unit 140 and the light emitting unit 150 are mounted on the substrate 160 (sensor substrate) at a predetermined interval. The light emitting unit 150 emits light to a test subject when driving power is supplied to the light emitting unit 150, and the light receiving unit 140 receives light (reflected light, transmitted light, or the like) from the test subject when driving power is supplied to the light receiving unit 140. For example, when the light emitting unit 150 emits light and the light is reflected by a test subject (for example, a blood vessel), the light receiving unit 140 receives and detects the reflected light. The light emitting unit 150 can be realized by a light receiving element such as an LED. The light receiving unit 140 can be realized by a light receiving element such as a photodiode, or a diode element of a PN junction which is formed on a semiconductor substrate, or the like. In this case, an angle limiting filter for narrowing a light reception angle or a wavelength limiting filter (optical filter film) that limits a wavelength of light incident on a light receiving element may be formed on the diode element.

Meanwhile, a dome-type lens 151 (condensing lens in a broad sense) which is provided in the light emitting unit 150 is a lens for condensing light from an LED chip (light emitting element chip in a broad sense) which is resin-sealed (sealed with a light transmitting resin) in the light emitting unit 150. That is, in the light emitting unit 150 which is a surface-mounted type, the LED chip is disposed below the dome-type lens 151, and light from the LED chip is condensed by the dome-type lens 151 and is emitted to a test subject. Thereby, it is possible to improve the optical efficiency of the light detection unit.

When a pulsimeter is taken as an example of the biological information measuring apparatus 1, light from the light emitting unit 150 travels within a test subject which is an object, and is diffused or scattered to epidermis, dermis, subcutaneous tissue, and the like. Thereafter, the light reaches a blood vessel (part to be detected) and is reflected. At this time, a portion of the light is absorbed into the blood vessel. Since the absorption of the light at the blood vessel varies by the influence of pulses and the amount of reflected light also varies, the light receiving unit 140 receives the reflected light and detects variations in the amount of light, and thus it is possible to detect a pulse rate which is biological information, and the like.

The light shielding member 70 (light shielding wall 100) as a light shielding unit is provided between the light receiving unit 140 and the light emitting unit 150. The light shielding member 70 (light shielding wall 100) prevents light from, for example, the light emitting unit 150 (direct light or the like) from being directly incident on the light receiving unit 140. The light shielding member 70 (light shielding wall 100) can be formed by, for example, sheet metal working. Meanwhile, an example of a material of the light shielding member 70 (light shielding wall 100) includes a resin such as rubber (including a natural resin and a synthetic resin) as a material other than a metal material.

The light shielding member 70 as a light shielding unit is a member for shielding light. In this embodiment, the light shielding member 70 is provided between the light receiving unit 140 and the light emitting unit 150 as the light shielding wall 100, and shields the light receiving unit 140. Meanwhile, the light shielding member 70 may be provided so as to cover a portion of the light receiving unit 140, and may be configured to shield light incident on the light receiving unit 140. It is possible to improve detection performance while preventing light (direct light) from the light emitting unit 150 from being incident on the light receiving unit 140, by the light shielding member 70 (light shielding wall 100).

In addition, it is preferable to perform a reflection suppressing process on at least the surface of the light receiving unit 140 on the side of the light shielding member 70 (light shielding wall 100) as a light shielding unit. For example, the light shielding member 70 is configured to have a surface (inner surface or the like) having a predetermined color such as a black color so that the irregular reflection of light is prevented. Alternatively, the light shielding member 70 may be configured to have a surface having a moth-eye structure. For example, a concavo-convex structure having several tens to several hundreds of cycles is formed in the surface of the light shielding member so as to configure a reflection preventing structure. When such a reflection suppressing process is performed, it is possible to effectively suppress the occurrence of a situation in which, for example, reflected light on the surface of the light shielding member 70 changes to stray light and becomes a noise component of a detection signal.

The light receiving unit 140, the light emitting unit 150, and the light shielding member 70 (light shielding wall 100) as a light shielding unit are mounted on the substrate 160. The substrate 160 is, for example, a rigid substrate. The substrate 160 is provided with a terminal (not shown) for connection to a terminal (not shown) of a signal and a power supply of the light receiving unit 140 and a terminal (not shown) for connection to a signal and a power supply of an external main substrate. For example, the terminal of the light receiving unit 140 and the terminal of the substrate 160 are connected to each other by wire bonding or the like.

In addition, the sensor unit 40 is provided with the throttle portion 80 (80a, 80b). The throttle portion 80 narrows light from a test subject in a light path between the test subject and the sensor unit 40, and narrows light from the light emitting unit 150. In FIG. 5, the throttle portion 80 is provided between the light transmitting member 50 and the light emitting unit 150. Here, the throttle portion 80 may be provided between the light transmitting member 50 and a test subject or within the light transmitting member 50.

The light transmitting member 50 is provided on a surface of the biological information measuring apparatus which comes into contact with a test subject, and transmits light from the test subject. In addition, the light transmitting member 50 comes into contact with the test subject when biological information on the test subject is measured. For example, the convex portion 52 (detection window) of the light transmitting member 50 comes into contact with the test subject. Meanwhile, it is preferable that the shape of the surface of the convex portion 52 is a curved surface shape (spherical shape). However, the invention is not limited thereto, and various shapes can be adopted. In addition, the light transmitting member 50 may be a member capable of transmitting a wavelength of light from a test subject, and a transparent material or a colored material may be used.

The groove portion 54 for suppressing a pressing fluctuation or the like is provided in the vicinity of the convex portion 52 of the light transmitting member 50. In addition, when a surface of the light transmitting member 50 which is provided with the convex portion 52 is set to be a first surface, the light transmitting member 50 has the concave portion 56 at a position corresponding to the convex portion 52 in a second surface on the back side of the first surface. The light receiving unit 140, the light emitting unit 150, the light shielding member 70, and the throttle portion 80 are provided in a space of the concave portion 56.

In addition, the pressing suppressing portion 58 that suppresses pressing applied to a test subject (skin of a wrist) by the convex portion 52 is provided on a surface of the biological information measuring apparatus on a test subject side. In FIG. 5, the pressing suppressing portion 58 is provided so as to surround the convex portion 52 of the light transmitting member 50. The convex portion 52 protrudes toward the test subject side further than a pressing suppressing portion (pressing suppressing surface) 58.

It is possible to apply initial pressing for exceeding, for example, a vein vanishing point to a test subject by providing the convex portion 52. In addition, the pressing suppressing portion 58 for suppressing pressing applied to the test subject by the convex portion 52 is provided, and thus it is possible to minimally suppress a pressing fluctuation in a usage range in which the measurement of biological information is performed by the biological information measuring apparatus and to achieve a reduction in a noise component and the like. In addition, when the convex portion 52 protrudes from the pressing suppressing portion 58, the convex portion 52 comes into contact with the test subject and applies initial pressing, and then the pressing suppressing portion 58 comes into contact with the test subject, and thus it is possible to suppress pressing applied to the test subject by the convex portion 52. The wording “vein vanishing point” as used herein refers to a point in which a signal caused by a vein superimposed on a pulse wave signal vanishes or becomes smaller to the extent that the signal does not affect the measurement of pulse waves, when the convex portion 52 is brought into contact with the test subject and the strength of pressing is sequentially increased.

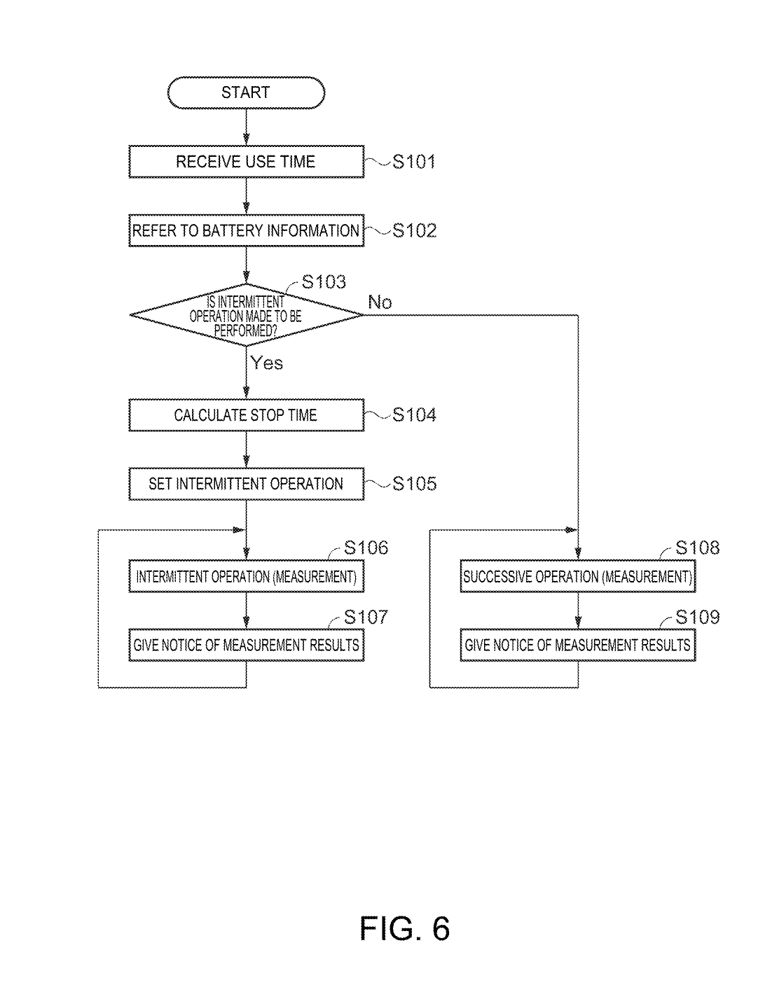

Next, a method of determining whether at least one of a light receiving unit and a light emitting unit is brought into an intermittent operation will be described with reference to FIG. 6.

FIG. 6 is a flow chart illustrating setting of whether a biological information measuring apparatus is brought into an intermittent operation.

First, in step S101, the biological information measuring apparatus 1 receives a use time. Specifically, a user inputs a use time using an input unit, and the control unit 200 of the biological information measuring apparatus 1 receives the input signal thereof. For example, the use time is a time until the secondary battery of the biological information measuring apparatus 1 is charged, or a time when a user desires to continue performing measurement. For example, the use time is input by causing a user to perform an operation, such as tapping or swiping, which is determined in advance on a touch panel (display unit 260).

In the next step S102, the biological information measuring apparatus 1 refers to battery information. Examples of the battery information include a charging amount, remaining amount, and consumption of a secondary battery. The control unit 200 of the biological information measuring apparatus 1 refers to at least one of a charging amount, remaining amount, and consumption of a secondary battery which are stored in the storage unit 240.

In step S103, the biological information measuring apparatus 1 performs determination of whether being brought into an intermittent operation. The control unit 200 calculates an operable time when a successive operation is made to be performed, on the basis of battery information, compares a use time which is input with the calculated operable time, and performs determination of whether being required to be brought into an intermittent operation. When the use time>the operable time, it is determined that the intermittent operation is required to be performed (S103: Yes), and the flow proceeds to step S104. When the use time<the operable time, it is determined that the intermittent operation is not required to be performed (S103: No), and the flow proceeds to step S108. The biological information measuring apparatus 1 performs successive measurement (successive operation) by continuously supplying driving power to a light receiving element of the light receiving unit 140 such as a photodiode and a light emitting element of the light emitting unit 150 such as an LED. In step S109, the control unit 200 notifies the display unit 260 of biological information, such as pulse waves, which is measured in step S108, and stores the data in the storage unit 240. Thereafter, the biological information measuring apparatus 1 repeatedly performs the successive operation in step S108 and the notification of biological information in step S109.

In step S104, the biological information measuring apparatus 1 calculates a stop time. The control unit 200 calculates conditions in which the relation of the use time<operable time is established by an intermittent operation being performed. In detail, in the intermittent operation, a measurement time when pulse waves are measured and a stop time when the measurement of pulse waves is stopped are alternately repeated. Regarding the measurement time, a minimum time required for one measurement of pulse waves is determined in advance, the control unit 200 calculates a stop time satisfying the relation of the use time<the operable time using the stop time as a parameter on the basis of the battery information and the measurement time.

In step S105, the biological information measuring apparatus 1 sets an intermittent operation. In the intermittent operation, a measurement time when pulse waves are measured and a stop time when the measurement of pulse waves is stopped are alternately repeated. The control unit 200 sets at least one of the stop time and the measurement time. In this embodiment, the control unit 200 sets the measurement time to be a minimum time which is determined in advance, and sets the stop time to be a time calculated in step S104.

In step S106, the biological information measuring apparatus 1 performs an intermittent operation. Specifically, driving power is supplied (ON) to the light receiving unit 140 and the light emitting unit 150 when a measurement time is set, and driving power is cut off (OFF) when a stop time is set. For example, the ON/OFF of driving power can be realized by providing a switch in a power supply line, not shown in the drawing, and causing the control unit 200 (see FIG. 4) to perform switch control in accordance with a stop time and a measurement time which are set.

In step S107, the biological information measuring apparatus 1 gives notice of measurement results. The control unit 200 notifies the display unit 260 of biological information, such as pulse waves, which is measured in step S106, and stores the data in the storage unit 240. Thereafter, the biological information measuring apparatus 1 repeatedly performs the intermittent operation in step S106 and the notice of measurement results in step S107.

After performing the above-described steps, the biological information measuring apparatus 1 determines whether at least one of the light receiving unit 140 and the light emitting unit 150 is brought into an intermittent operation on the basis of a use time which is input by a user and at least one of a charging amount, remaining amount, and consumption of a secondary battery. When it is determined that an intermittent operation is required, at least one of a stop time and a measurement time is set, the biological information measuring apparatus 1 performs an intermittent operation, and thus the power consumption of the biological information measuring apparatus 1 is reduced. Thereby, an operable time of the biological information measuring apparatus 1 is increased until a necessary use time according to the user's situation.

Meanwhile, in this embodiment, a description has been given on the assumption that driving power to both the light receiving unit 140 and the light emitting unit 150 is cut off, but there is an effect of reducing power consumption even in a configuration in which driving power to at least one of the light receiving unit 140 and the light emitting unit 150 is cut off.

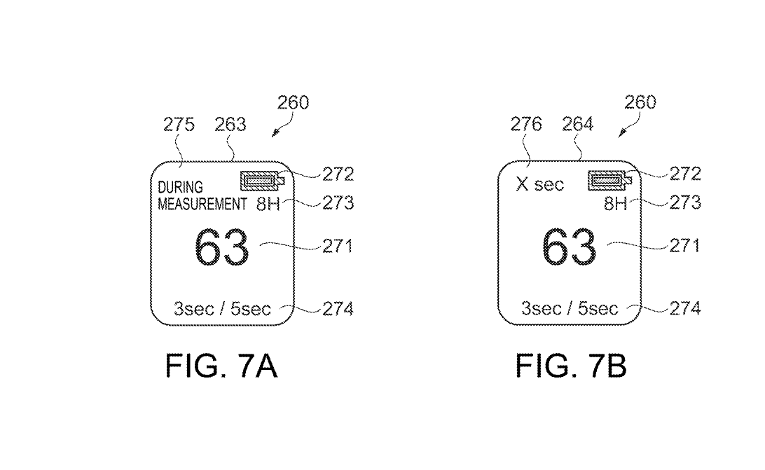

Next, a description will be given of display contents displayed on the display unit 260 in a state where the biological information measuring apparatus 1 is set to be in an intermittent operation mode. FIG. 7A illustrates a notification screen which is displayed at a measurement time, and FIG. 7B is a notification screen which is displayed at a stop time.

As illustrated in FIGS. 7A and 7B, each of notification screens 263 and 264 displayed on the display unit 260 includes a pulse display 271 that indicates a measured pulse rate (for example, “63”), a battery mark 272, an operable time display 273, and a set content display 274. The pulse display 271 gives notice of the measured pulse rate (for example, “63”) and is provided in the middle of each of the notification screens 263 and 264.

The battery mark 272 indicates a remaining amount of a secondary battery, and is provided, for example, on the upper right of each of the notification screens 263 and 264. The operable time display 273 indicates an operable time (for example, “8H”) for which the biological information measuring apparatus 1 can be brought into operation. For example, it is possible to intuitively inform that “8H” indicates an operable time, by displaying the operable time display 273 and the battery mark 272 in a combined manner. In addition, an operable time is always displayed on the notification screens 263 and 264, and thus a user can prevent the continuous recording of biological information from being stopped due to forgetfulness of charging a secondary battery, or the like.

The control unit 200 of the biological information measuring apparatus 1 notifies the display unit 260 that switching from a successive operation to an intermittent operation has been performed. The set content display 274 indicates contents of an intermittent operation which is set, and is provided, for example, on the lower side in each of the notification screens 263 and 264. The contents of the intermittent operation can be expressed by a measurement time and a stop time. For example, in this embodiment, a notice is given through a measurement time/a stop time (for example, “3 sec/5 sec”). Simultaneously with this, a user may be notified through vibration by driving the vibration generating unit 180. Thereby, a user can always ascertain the switching of the biological information measuring apparatus 1 to an intermittent operation and the contents of the intermittent operation, and can determine whether the set contents are appropriate in the current action (for example, during running).

As illustrated in FIG. 7A, the notification screen 263 displayed on the display unit 260 includes a display during measurement 275. The display during measurement 275 indicates a measurement mode in which biological information is being measured, during a measurement time, and displays characters such as “During Measurement”. Thereby, a user can recognize the time when the biological information measuring apparatus 1 is performing measurement during an intermittent operation. Meanwhile, the display of characters of “During Measurement” displayed in the display during measurement 275 is illustrative, and the display may be performed using a sign or a pictograph.

As illustrated in FIG. 7B, the notification screen 264 displayed on the display unit 260 includes a display during stop 276. The display during stop 276 gives notice of a time until the measurement of biological information is started, during a stop time. In this embodiment, the display is performed in a count-down mode. Thereby, a user can prevent an abnormal value from being measured due to a sudden body motion or the like. In addition, in this embodiment, the display during measurement 275 and the display during stop 276 are displayed at the same position. Thereby, a user can easily ascertain whether the biological information measuring apparatus 1 is in a measurement time mode or a stop time mode in an intermittent operation.

Meanwhile, in this embodiment, a description has been given on the assumption that the control unit 200 fixes a measurement time to a minimum time which is determined in advance and calculates a stop time satisfying the relation of a use time<an operable time using the stop time as a parameter, but the control unit may obtain conditions in which the relation of a use time<an operable time is established by using both the measurement time and the stop time as parameters to thereby perform an intermittent operation.

In addition, in this embodiment, a description has been given on the assumption that an input operation is performed by tapping or swiping a touch panel (display unit 260), but the invention is not limited thereto. The input may be performed through an operation such as long tapping or flicking.

In addition, a display form or a display location of each of the pulse display 271, the battery mark 272, the operable time display 273, the set content display 274, the display during measurement 275, and the display during stop 276 which is displayed on the display unit 260 is illustrative, and the invention is not limited thereto.

As described above, according to the biological information measuring apparatus 1 of this embodiment, the following effects can be obtained.

The control unit 200 of the biological information measuring apparatus 1 determines whether at least one of the light receiving unit 140 and the light emitting unit 150 is brought into an intermittent operation on the basis of a use time which is input according to a user's situation and at least one of a charging amount, remaining amount, and consumption of a secondary battery which are stored in the storage unit 240. The biological information measuring apparatus 1 performs an intermittent operation by setting at least one of a stop time and a measurement time. Thereby, the power consumption of the biological information measuring apparatus 1 is reduced. Therefore, it is possible to provide the biological information measuring apparatus of which the operable time is automatically increased.

In addition, the control unit 200 of the biological information measuring apparatus 1 displays contents of an intermittent operation in the set content display 274 of the display unit 260 when switching from a successive operation to the intermittent operation is performed. Thereby, a user can always ascertain the biological information measuring apparatus 1 being in an intermittent operation mode and the contents of the intermittent operation and can determine whether the set contents are appropriate.

In addition, the control unit 200 of the biological information measuring apparatus 1 includes the display during measurement 275 notifying the display unit 260 that biological information is being measured during an intermittent operation, and thus a user can recognize the time when the biological information measuring apparatus 1 is performing measurement during an intermittent operation.

In addition, the control unit 200 of the biological information measuring apparatus 1 includes the operable time display 273 notifying the display unit 260 of an operable time, and thus a user can prevent the continuous recording of biological information from being stopped due to forgetfulness of charging a secondary battery, or the like.

### Second Embodiment

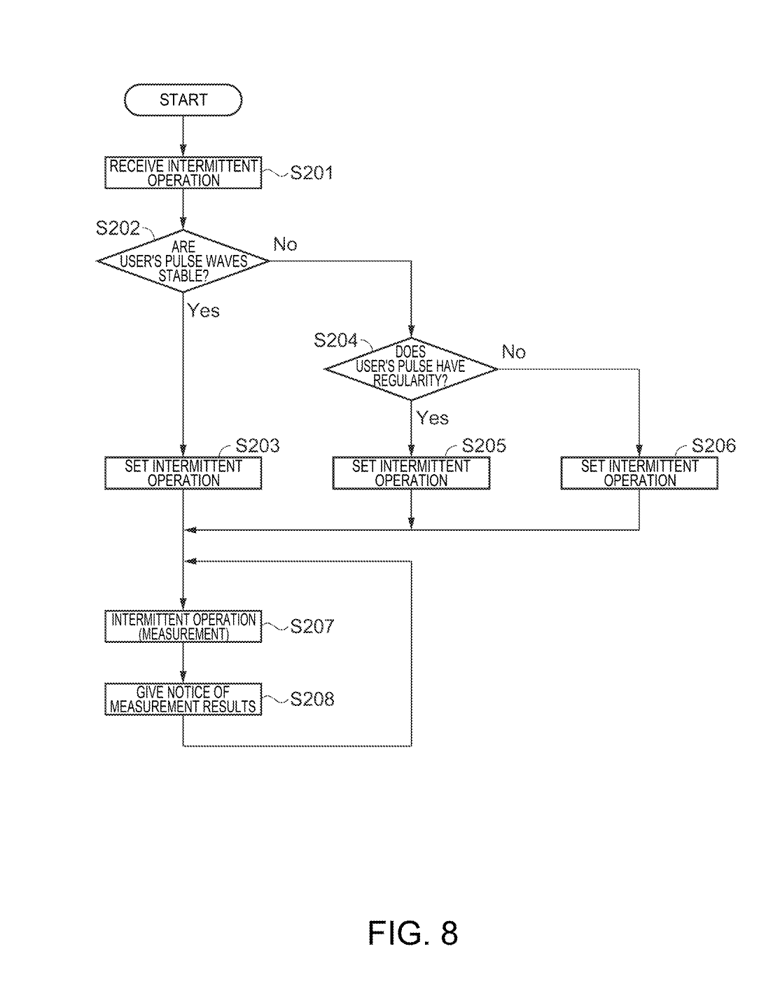

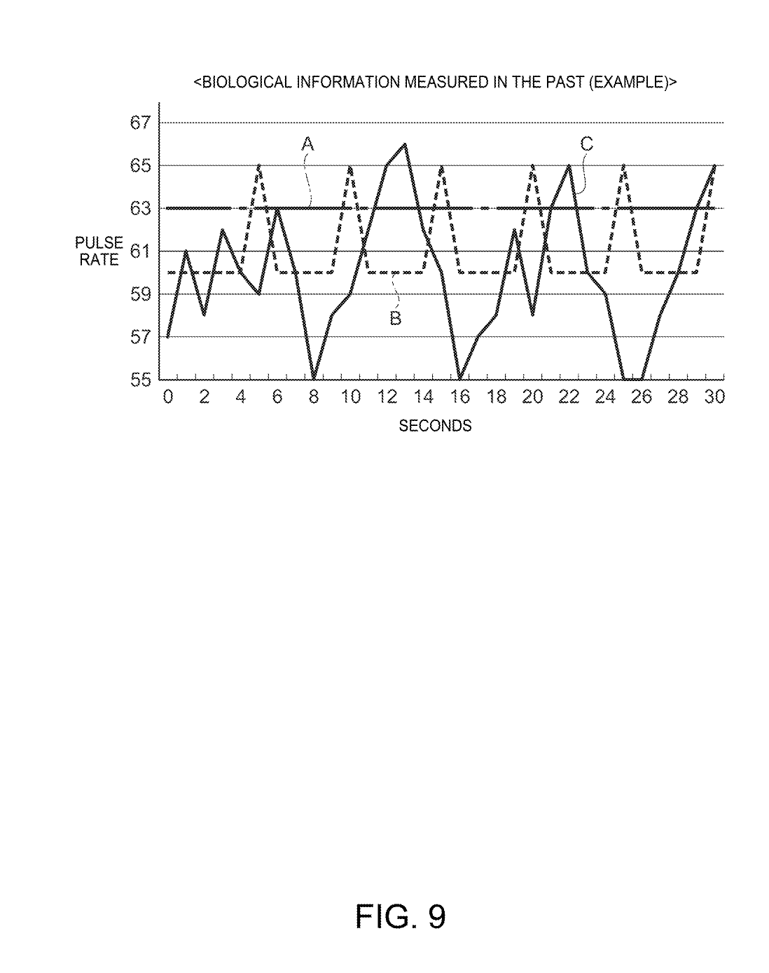

FIG. 8 is a flow chart illustrating an intermittent operation of a biological information measuring apparatus according to a second embodiment. FIG. 9 is a diagram illustrating an example of biological information measured in the past. The biological information measuring apparatus according to this embodiment will be described with reference to these drawings. Meanwhile, the same components as those in the first embodiment are denoted by the same reference numerals and signs, and a repeated description will be omitted. In a biological information measuring apparatus 2 according to this embodiment, at least one of a stop time and a measurement time is set on the basis of biological information which is measured in the past.

First, in step S201, the biological information measuring apparatus 2 receives an intermittent operation. Specifically, a user inputs an intermittent operation of the biological information measuring apparatus 2 using an input unit, and a control unit 200 of the biological information measuring apparatus 2 receives the input signal. For example, the intermittent operation is input by causing a user to perform an operation, such as tapping or swiping, which is determined in advance on a touch panel (display unit 260).

In the next step S202, the biological information measuring apparatus 2 determines whether user's pulse waves are stable. In detail, the control unit 200 of the biological information measuring apparatus 2 refers to measurement results of biological information, measured in the past, which are stored in a storage unit 240. FIG. 9 illustrates an example of measurement results of biological information.

FIG. 9 is a diagram illustrating pulse waves (pulse rate) as biological information measured in the past. A horizontal axis represents a measurement time, and a vertical axis represents a pulse rate. In FIG. 9, pieces of biological information on three users A, B, and C are displayed as examples.

A pulse rate of the user A indicates a case of being “63”, which is stable. The pulse of the user B indicates a case of a singular point that a pulse rate rises to “65” regularly. The pulse of the user C indicates a case of fluctuating irregularly all the time.

For example, in the case of the user A, it is determined that the pulse rate is stable (S202: Yes), and the flow proceeds to step S203. In the cases of the users B and C, it is determined that the pulse rate is unstable (S202: No), and the flow proceeds to step S204.

In step S203, the biological information measuring apparatus 2 sets an intermittent operation. In the intermittent operation, a measurement time when pulse waves are measured and a stop time when the measurement of pulse waves is stopped are alternately repeated. The stop time of the biological information measuring apparatus 2 according to this embodiment is set to a long stop time (for example, 20 seconds) which is determined in advance, a short stop time (for example, 5 seconds), and an intermediate stop time (for example, 10 seconds) between the long stop time and the short stop time. Since the pulse rate of the user A is stable, the control unit 200 sets the stop time to a long stop time. Meanwhile, the measurement time is set to a time which is determined in advance.

In step S204, the biological information measuring apparatus 2 determines whether a user′ pulse has regularity. For example, in the case of the user B, it is determined that the user's pulse has regularity (S204: Yes), and the flow proceeds to step S205. In the case of the user C, it is determined that the user's pulse does not have regularity (S204: No), and the flow proceeds to step S206.

In step S205, the biological information measuring apparatus 2 sets an intermittent operation. Since the pulse waves of the user B have a singular point for every five seconds, the control unit 200 sets a measurement time to a time longer than five seconds and sets a stop time to an intermediate stop time.

In step S206, the biological information measuring apparatus 2 sets an intermittent operation. Since the pulse waves of the user C always irregularly fluctuate, the control unit 200 sets a stop time to a short stop time.

In step S207, the biological information measuring apparatus 2 sets an intermittent operation. Specifically, driving power is supplied (ON) to a light receiving unit 140 and a light emitting unit 150 when a measurement time is set, and driving power is cut off (OFF) when a stop time is set. For example, the ON/OFF of driving power can be realized by providing a switch in a power supply line, not shown in the drawing, and causing the control unit 200 (see FIG. 4) to perform switch control in accordance with a stop time and a measurement time which are set.

In step S208, the biological information measuring apparatus 2 gives notice of measurement results. The control unit 200 notifies a display unit 260 of biological information, such as pulse waves, which is measured in step S207, and stores the data in the storage unit 240. Thereafter, the biological information measuring apparatus 2 repeatedly performs the intermittent operation in step S207 and the notice of measurement results in step S208.

As described above, according to the biological information measuring apparatus 2 of this embodiment, the following effects can be obtained.

The biological information measuring apparatus 2 performs an intermittent operation by setting at least one of a stop time and a measurement time on the basis of biological information which was measured in the past after performing the above-described steps, and thus the power consumption of the biological information measuring apparatus 2 is reduced. Thereby, an operable time of the biological information measuring apparatus 2 is increased.

Meanwhile, a description has been given on the assumption that biological information measured in the past is stored in the storage unit 240, but the biological information may be stored in a database which is provided outside. When an external database is used, data (biological information) is stored and referred to through wireless communication of a communication unit 250 (see FIG. 4).

### Third Embodiment

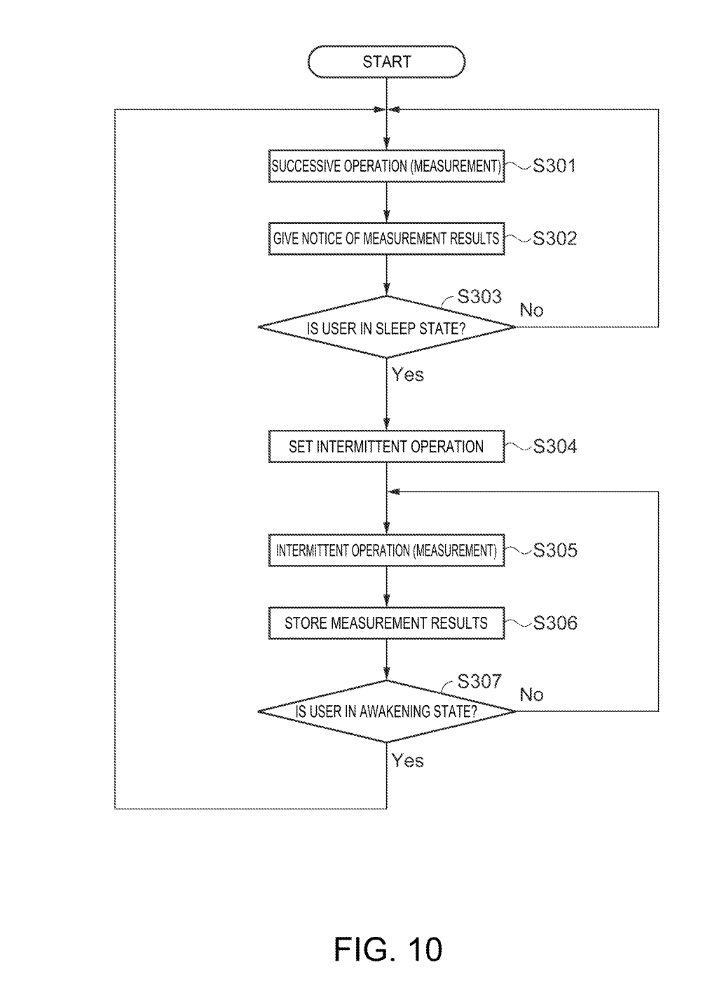

FIG. 10 is a flowchart illustrating an intermittent operation of a biological information measuring apparatus according to a third embodiment. The biological information measuring apparatus according to this embodiment will be described with reference to FIG. 10. Meanwhile, the same components as those in the first embodiment are denoted by the same reference numerals and signs, and a repeated description will be omitted. A biological information measuring apparatus 3 according to this embodiment is set to be in an intermittent operation mode when it is determined that a user is in a sleep state.

In step S301, the biological information measuring apparatus 3 performs a successive operation. The biological information measuring apparatus 3 performs successive measurement (successive operation) by continuously supplying driving power to a light receiving element of a light receiving unit 140 such as a photodiode and a light emitting element of a light emitting unit 150 such as an LED. At this time, a control unit 200 (see FIG. 4) receives a body motion detection signal which is output from a body motion sensor unit 170 to thereby obtain the amount of body motion of a user.

In step S302, the biological information measuring apparatus 3 gives notice of measurement results. The control unit 200 of the biological information measuring apparatus 3 notifies a display unit 260 of biological information, such as pulse waves, which is measured in step S301, and stores the data in a storage unit 240.

In step S303, the biological information measuring apparatus 3 determines whether a user is in a sleep state. When the amount of body motion of the user which is obtained in step S301 and a variation in pulse waves are set to have a value equal to or less than a predetermined value, it is determined that the user is in a sleep state (S303: Yes), and the flow proceeds to step S304. When it is determined that the user is in an awakening state (S303: No), the flow returns to step S301 to continue performing successive measurement.

In step S304, the biological information measuring apparatus 3 sets an intermittent operation. In the intermittent operation, a measurement time when pulse waves are measured and a stop time when the measurement of pulse waves is stopped are alternately repeated. Since pulse waves of a user in a sleep state are stable, the control unit 200 sets a measurement time and a stop time of an intermittent operation to a time which is determined in advance.

In step S305, the biological information measuring apparatus 3 performs an intermittent operation. Specifically, driving power is supplied (ON) to the light receiving unit 140 and the light emitting unit 150 when a measurement time is set, and driving power is cut off (OFF) when a stop time is set. For example, the ON/OFF of driving power can be realized by providing a switch in a power supply line, not shown in the drawing, and causing the control unit 200 to perform switch control in accordance with a stop time and a measurement time which are set. At this time, the control unit 200 receives a body motion detection signal which is output from a body motion sensor unit 170 to thereby obtain the amount of body motion of a user.

In step S306, the biological information measuring apparatus 3 stores measurement results. The control unit 200 notifies the display unit 260 of biological information, such as pulse waves, which is measured in step S305, and stores the data in the storage unit 240. Meanwhile, when a user is in a sleep state, the display unit 260 may be set to be in a non-display state. Thereby, it is possible to reduce the power consumption of the biological information measuring apparatus 3.

In step S307, the biological information measuring apparatus 3 determines whether a user is in an awakening state. When it is determined that the user is in an awakening state (S307: Yes) because the amount of body motion of the user which is obtained in step S305 and a variation in pulse waves are set to have a value equal to or greater than a predetermined value, the control unit 200 sets a successive operation, and the flow returns to step S301 and proceeds to successive measurement. When it is determined that the user is in a sleep state (S307: No), the flow returns to step S305 to continue performing an intermittent operation.

As described above, according to the biological information measuring apparatus 3 of this embodiment, the following effects can be obtained.

The control unit 200 of the biological information measuring apparatus 3 sets and performs an intermittent operation when it is determined that a user is in a sleep state after performing the above-described steps, and thus the power consumption of the biological information measuring apparatus 3 is reduced. Thereby, an operable time of the biological information measuring apparatus 3 is increased.

In addition, the biological information measuring apparatus 3 includes an acceleration sensor 172, and thus can detect the state (sleep state, awakening state, or the like) of a user.

### Fourth Embodiment

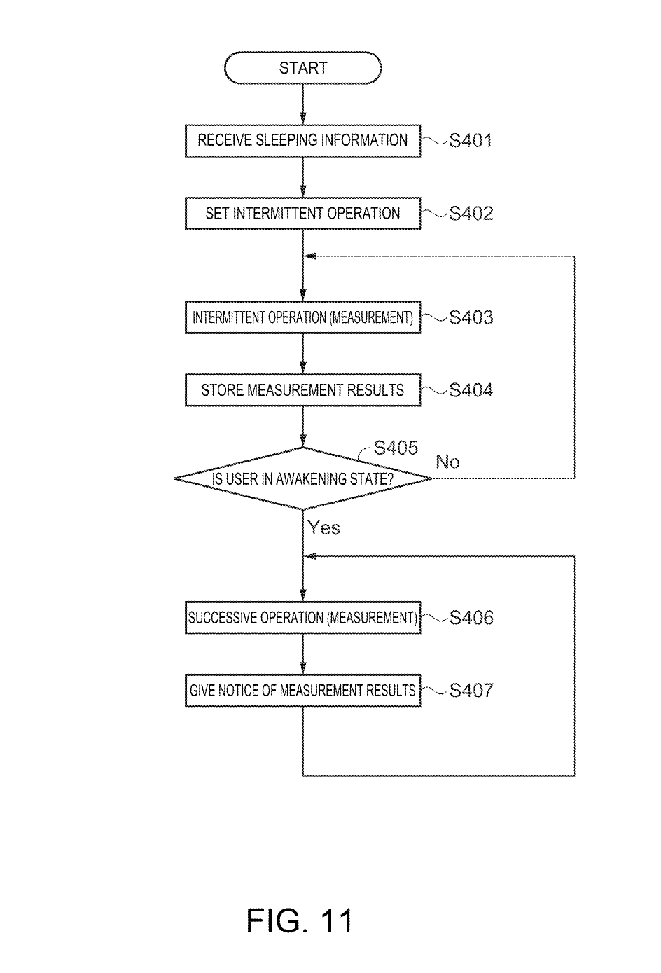

FIG. 11 is a flow chart illustrating an intermittent operation of a biological information measuring apparatus according to a fourth embodiment. The biological information measuring apparatus according to this embodiment will be described with reference to FIG. 11. Meanwhile, the same components as those in the first embodiment are denoted by the same reference numerals and signs, and a repeated description will be omitted. A biological information measuring apparatus 4 according to this embodiment is set to be in an intermittent operation mode when a user inputs sleeping information.

First, in step S401, the biological information measuring apparatus 4 receives sleeping information. Specifically, a user inputs a sleeping mode using an input unit, and a control unit 200 (see FIG. 4) of the biological information measuring apparatus 4 receives the input signal thereof. The sleeping information is input by causing a user to perform an operation, such as tapping or swiping, which is determined in advance on a touch panel (display unit 260).

In step S402, the biological information measuring apparatus 4 sets an intermittent operation. In the intermittent operation, a measurement time when pulse waves are measured and a stop time when the measurement of pulse waves is stopped are alternately repeated. Since pulse waves of a user in a sleeping (sleep) state are stable, the control unit 200 sets a measurement time and a stop time of an intermittent operation to a time which is determined in advance.

In step S403, the biological information measuring apparatus 4 performs an intermittent operation. Specifically, driving power is supplied (ON) to a light receiving unit 140 and a light emitting unit 150 when a measurement time is set, and driving power is cut off (OFF) when a stop time is set. For example, the ON/OFF of driving power can be realized by providing a switch in a power supply line, not shown in the drawing, and causing the control unit 200 to perform switch control in accordance with a stop time and a measurement time which are set. At this time, the control unit 200 receives a body motion detection signal which is output from a body motion sensor unit 170 to thereby obtain the amount of body motion of a user.

In step S404, the biological information measuring apparatus 4 stores measurement results. The control unit 200 notifies the display unit 260 of biological information, such as pulse waves, which is measured in step S403, and stores the data in a storage unit 240. Meanwhile, when a user is in a sleep state, the display unit 260 may be set to be in a non-display state. Thereby, it is possible to reduce the power consumption of the biological information measuring apparatus 4.

In step S405, the biological information measuring apparatus 4 determines whether a user is in an awakening state. When it is determined that the user is in an awakening state (S405: Yes) because the amount of body motion of the user which is obtained in step S403 and a variation in pulse waves are set to have a value equal to or greater than a predetermined value, the control unit 200 sets a successive operation, and the flow proceeds to step S406. When it is determined that the user is in a sleep state (S405: No), the flow returns to step S403 to continue performing an intermittent operation.

In step S406, the biological information measuring apparatus 4 performs a successive operation. The biological information measuring apparatus 4 performs successive measurement (successive operation) by continuously supplying driving power to a light receiving element of the light receiving unit 140 such as a photodiode and a light emitting element of the light emitting unit 150 such as an LED.

In step S407, the biological information measuring apparatus 4 gives notice of measurement results. The control unit 200 of the biological information measuring apparatus 4 notifies the display unit 260 of biological information, such as pulse waves, which is measured in step S406, and stores the data in the storage unit 240. Thereafter, the biological information measuring apparatus 4 repeatedly performs the processes of steps S406 and S407.

As described above, according to the biological information measuring apparatus 4 of this embodiment, the following effects can be obtained.

The control unit 200 of the biological information measuring apparatus 4 sets and performs an intermittent operation when a user's sleeping information is received after performing the above-described steps, and thus the power consumption of the biological information measuring apparatus 4 is reduced. Thereby, an operable time of the biological information measuring apparatus 4 is increased.

### Fifth Embodiment

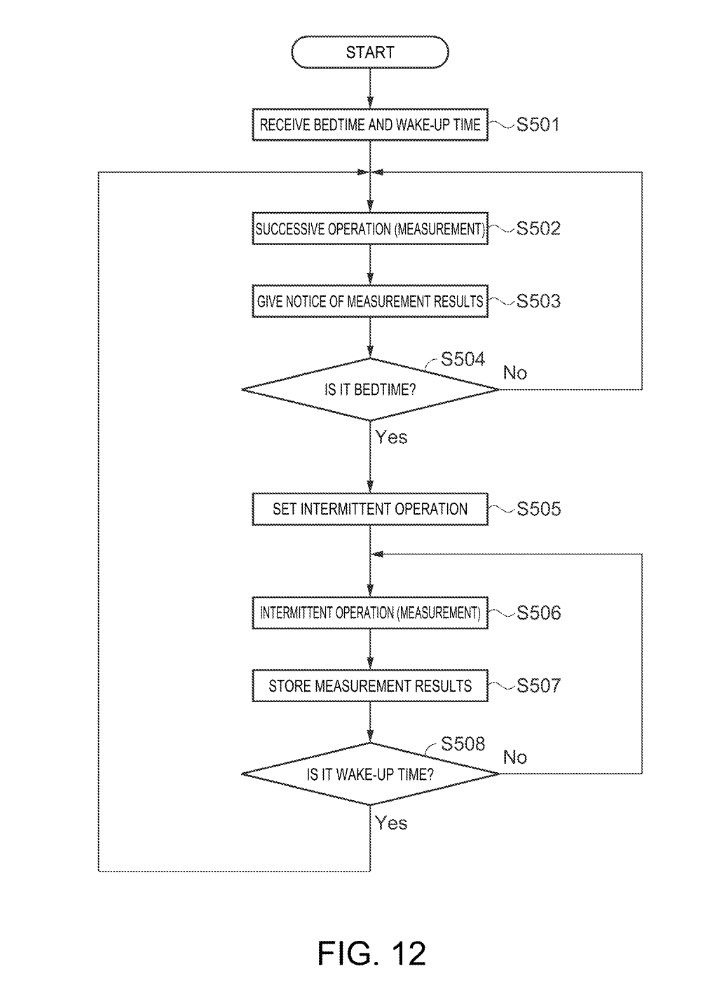

FIG. 12 is a flow chart illustrating an intermittent operation of a biological information measuring apparatus according to a fifth embodiment. The biological information measuring apparatus according to this embodiment will be described with reference to FIG. 12. Meanwhile, the same components as those in the first embodiment are denoted by the same reference numerals and signs, and a repeated description will be omitted. A biological information measuring apparatus 5 according to this embodiment is set to be in an intermittent operation mode in accordance with a bedtime and a wake-up time which are input by a user.

First, in step S501, the biological information measuring apparatus 5 receives a bedtime and a wake-up time. Specifically, a user inputs a time when he or she goes to bed and a time when he or she wakes up using an input unit, and a control unit 200 (see FIG. 4) of the biological information measuring apparatus 5 receives the input signal thereof. For example, the bedtime and the wake-up time are input by causing a user to perform an operation, such as tapping or swiping, which is determined in advance on a touch panel (display unit 260) and to select a time displayed on the display unit 260.

In step S502, the biological information measuring apparatus 5 performs a successive operation. The biological information measuring apparatus 5 performs successive measurement (successive operation) by continuously supplying driving power to a light receiving element of a light receiving unit 140 such as a photodiode and a light emitting element of a light emitting unit 150 such as an LED.

In step S503, the biological information measuring apparatus 5 gives notice of measurement results. The control unit 200 of the biological information measuring apparatus 5 notifies the display unit 260 of biological information, such as pulse waves, which is measured in step S502, and stores the data in the storage unit 240.

In step S504, the biological information measuring apparatus 5 performs determination of whether being a bedtime. The control unit 200 compares the present time with the bedtime received in step S501. When the present time has passed the bedtime (S504: Yes), the flow proceeds to step S505. When the present time is earlier than the bedtime (S504: No), the flow returns to step S502 to continue performing successive measurement.

In step S505, the biological information measuring apparatus 5 sets an intermittent operation. In the intermittent operation, a measurement time when pulse waves are measured and a stop time when the measurement of pulse waves is stopped are alternately repeated. Since pulse waves of a user in a sleeping (sleep) state are stable, the control unit 200 sets a measurement time and a stop time of an intermittent operation to a time which is determined in advance.

In step S506, the biological information measuring apparatus 5 performs an intermittent operation. Specifically, driving power is supplied (ON) to the light receiving unit 140 and the light emitting unit 150 when a measurement time is set, and driving power is cut off (OFF) when a stop time is set. For example, the ON/OFF of driving power can be realized by providing a switch in a power supply line, not shown in the drawing, and causing the control unit 200 to perform switch control in accordance with a stop time and a measurement time which are set.

In step S507, the biological information measuring apparatus 5 stores measurement results. The control unit 200 notifies the display unit 260 of biological information, such as pulse waves, which is measured in step S506, and stores the data in a storage unit 240. Meanwhile, when a user is in a sleep state, the display unit 260 may be set to be in a non-display state. Thereby, it is possible to reduce the power consumption of the biological information measuring apparatus 5.

In step S508, the biological information measuring apparatus 5 performs determination of whether being a wake-up time. The control unit 200 compares the present time with the wake-up time received in step S501. When the present time has passed the wake-up time (S508: Yes), the control unit sets a successive operation. The flow returns to step S502, and the processes of steps S502 to S508 are repeatedly performed. A vibration generating unit 180 (see FIG. 4) may be driven at the wake-up time as a wake-up alarm. When the present time is earlier than the wake-up time (S508: No), the flow returns to step S506 to continue performing an intermittent operation.

As described above, according to the biological information measuring apparatus 5 of this embodiment, the following effects can be obtained.

The control unit 200 of the biological information measuring apparatus 5 sets and performs an intermittent operation in accordance with a bedtime and a wake-up time which are input by a user after performing the above-described steps, and thus the power consumption of the biological information measuring apparatus 5 is reduced. Thereby, an operable time of the biological information measuring apparatus 5 is increased.

### Sixth Embodiment

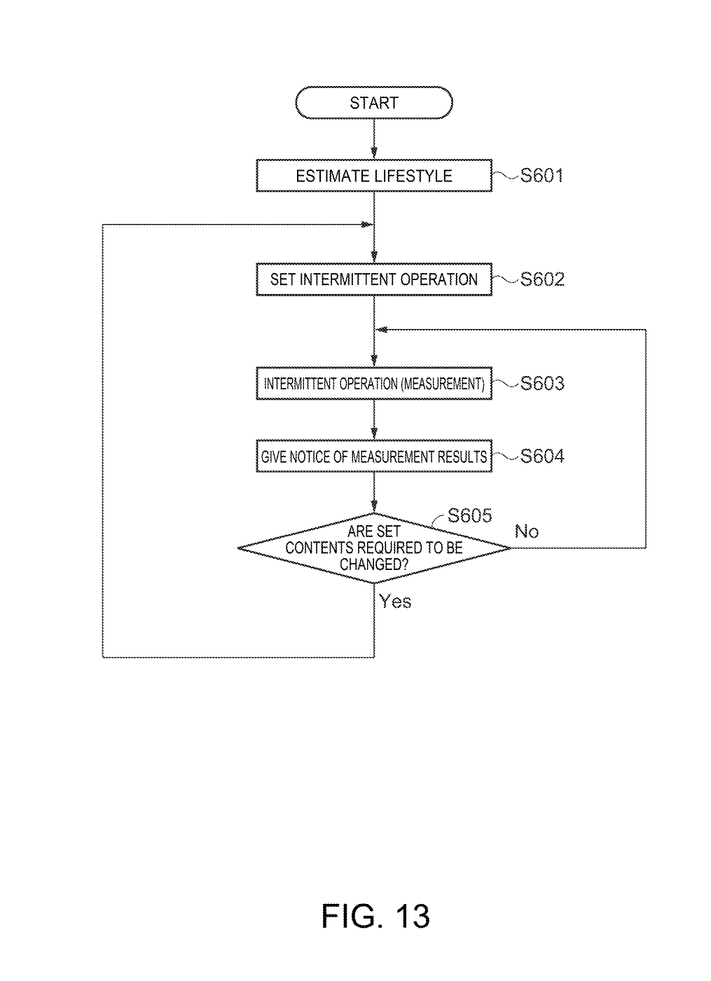

FIG. 13 is a flow chart illustrating an intermittent operation of a biological information measuring apparatus according to a sixth embodiment. The biological information measuring apparatus according to this embodiment will be described with reference to FIG. 13. Meanwhile, the same components as those in the first embodiment are denoted by the same reference numerals and signs, and a repeated description will be omitted. A biological information measuring apparatus 6 according to this embodiment is set to be in an intermittent operation mode on the basis of a user's lifestyle which is determined by biological information of the past when data of user's biological information is sufficiently stored.

In step S601, the biological information measuring apparatus 6 estimates lifestyle. In detail, a control unit 200 of the biological information measuring apparatus 6 refers to measurement results of biological information, measured in the past, which are stored in a storage unit 240. As the measurement results, data such as “activity level” (see FIG. 24) to be described later can be used. The “activity level” is represented by three activity levels of “high”, “middle”, and “low” with respect to, for example, a predetermined unit time by lifestyle such as a user's bedtime, desk work time, or exercise time and measured biological information. The control unit 200 estimates a user's lifestyle for today with reference to, for example, data of the same day of the week which was measured in the past. That is, an activity table showing an activity level with respect to a time is generated.

In step S602, the biological information measuring apparatus 6 sets an intermittent operation. In the intermittent operation, a measurement time when pulse waves are measured and a stop time when the measurement of pulse waves is stopped are alternately repeated. The control unit 200 sets a stop time and a measurement time with reference to the activity table generated in step S601 and the present time. The stop time is set to a short stop time (for example, 5 seconds) which is determined in advance when an activity level is “high”, is set to a long stop time (for example, 20 seconds) when an activity level is “low”, and is set to an intermediate stop time (for example, 10 seconds) between the long stop time and the short stop time when an activity level is “middle” in accordance with the present activity level. The measurement time is set to a time which is determined in advance.

In step S603, the biological information measuring apparatus 6 performs an intermittent operation. Specifically, driving power is supplied (ON) to a light receiving unit 140 and a light emitting unit 150 when a measurement time is set, and driving power is cut off (OFF) when a stop time is set. For example, the ON/OFF of driving power can be realized by providing a switch in a power supply line, not shown in the drawing, and causing the control unit 200 to perform switch control in accordance with a stop time and a measurement time which are set.

In step S604, the biological information measuring apparatus 6 gives notice of measurement results. The control unit 200 notifies a display unit 260 of biological information, such as pulse waves, which is measured in step S603, and stores the data in the storage unit 240.

In step S605, the biological information measuring apparatus 6 determines whether set contents are required to be changed. In other words, the control unit 200 determines whether an activity level used to set an intermittent operation is the same as an activity level at the present time with reference to the activity table generated in step S601, the present time, and set contents of the present intermittent operation. When the activity level used to set an intermittent operation is the same as the activity level at the present time, it is determined that set contents are not required to be changed (S605: No), and the flow returns to step S603 to repeatedly perform the intermittent operation. When the activity level used to set an intermittent operation is not the same as the activity level at the present time, it is determined that set contents are required to be changed (S605: Yes), and the flow returns to step S602 to set an intermittent operation again.

As described above, according to the biological information measuring apparatus 6 of this embodiment, the following effects can be obtained.

The control unit 200 of the biological information measuring apparatus 6 sets and performs an intermittent operation on the basis of a user's lifestyle which is determined by biological information of the past after performing the above-described steps, and thus the power consumption of the biological information measuring apparatus 6 is reduced. Thereby, an operable time of the biological information measuring apparatus 6 is increased.

### Seventh Embodiment

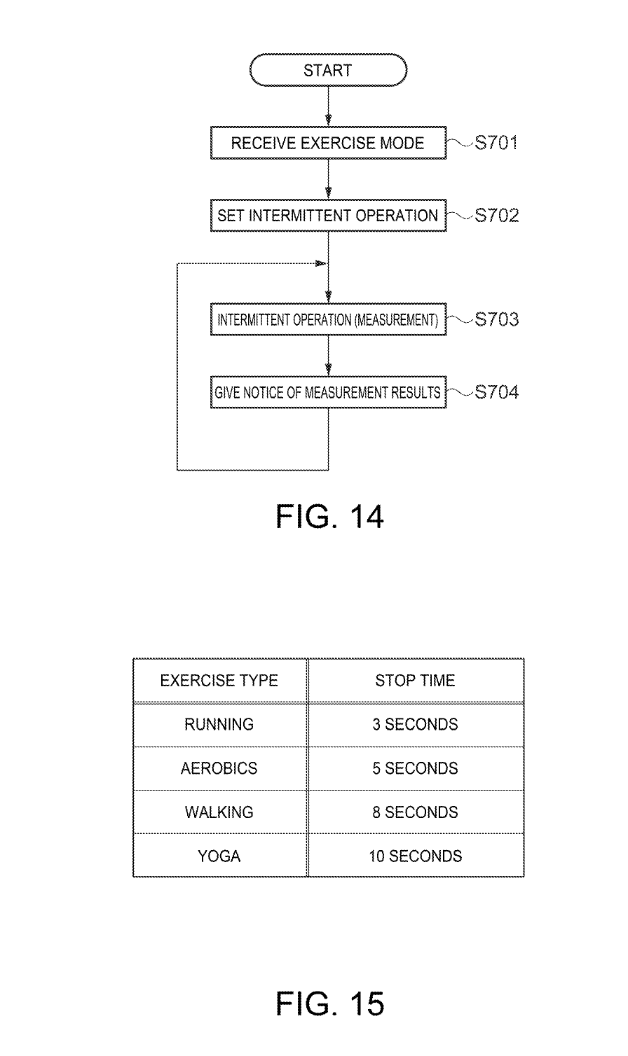

FIG. 14 is a flowchart illustrating an intermittent operation of a biological information measuring apparatus according to a seventh embodiment. FIG. 15 is a table illustrating stop times according to exercise types. A biological information measuring apparatus according to this embodiment will be described with reference to the accompanying drawings. Meanwhile, the same components as those in the first embodiment are denoted by the same reference numerals and signs, and a repeated description will be omitted. In a biological information measuring apparatus 7 according to this embodiment, an intermittent operation is set in accordance with an exercise type which is input by a user.

First, in step S701, the biological information measuring apparatus 7 receives an exercise mode. Specifically, a user inputs the start of exercise (exercise mode) and an exercise type using an input unit, and a control unit 200 of the biological information measuring apparatus 7 receives the input signal thereof. For example, the exercise mode and the exercise type are input by causing a user to perform an operation, such as tapping or swiping, which is determined in advance on a touch panel (display unit 260) and to select a selection item displayed on the display unit 260.

In step S702, the biological information measuring apparatus 7 sets an intermittent operation. In the intermittent operation, a measurement time when pulse waves are measured and a stop time when the measurement of pulse waves is stopped are alternately repeated. The control unit 200 refers to a table showing stop times according to exercise types stored in a storage unit 240. The table is set on the basis of exercise intensity (exercise load applied to a user) which varies depending on an exercise type. A stop time is reduced at the time of taking exercise having high exercise intensity, and is increased at the time of taking exercise having low exercise intensity. Thereby, it is possible to timely measure a variation in biological information according to exercise intensity. An example of the table showing stop times according to exercise types is illustrated in FIG. 15. As illustrated in FIG. 15, in the table showing stop times according to exercise types, a stop time which is set at the time of performing an intermittent operation is determined in advance in accordance with an exercise type. The control unit 200 sets a stop time of an intermittent operation in accordance with the input exercise type. For example, when “running” is input, a stop time is set to “3 seconds”. Meanwhile, a measurement time is set to a time which is required for one measurement.

In step S703, the biological information measuring apparatus 7 performs an intermittent operation. Specifically, driving power is supplied (ON) to a light receiving unit 140 and a light emitting unit 150 when a measurement time is set, and driving power is cut off (OFF) when a stop time is set. For example, the ON/OFF of driving power can be realized by providing a switch in a power supply line, not shown in the drawing, and causing the control unit 200 to perform switch control in accordance with a stop time and a measurement time which are set.

In step S704, the biological information measuring apparatus 7 gives notice of measurement results. The control unit 200 notifies the display unit 260 of biological information, such as pulse waves, which is measured in step S703. Thereafter, the biological information measuring apparatus 7 repeatedly performs the processes of step S703 and step S704 until receiving a signal for cancelling an exercise mode or a signal for inputting another exercise type.

As described above, according to the biological information measuring apparatus 7 of this embodiment, the following effects can be obtained.

The control unit 200 of the biological information measuring apparatus 7 sets an intermittent operation in accordance with an exercise type which is input by a user and performs the intermittent operation after performing the above-described steps, and thus the power consumption of the biological information measuring apparatus 7 is reduced. Thereby, an operable time of the biological information measuring apparatus 7 is increased.

Meanwhile, in the first to seventh embodiments, a touch panel has been illustrated as an input unit, but the invention is not limited thereto. A button or a stem is provided in each of the biological information measuring apparatuses 1 to 7, and may be used as an input unit.

In addition, in the first to seventh embodiments, a description has been given on the assumption that data such as measured biological information or various types of tables is stored in the storage unit 240, but the data may be stored in a database which is provided outside. When an external database is used, data is stored and referred to through wireless communication or wired communication of a communication unit 250 (see FIG. 4).

In addition, an input to each of the biological information measuring apparatuses 1 to 7 may be performed using a portable communication terminal, an information processing terminal, or the like without being performed using an input unit in the main body, and may be performed by connecting a terminal and each of the biological information measuring apparatuses 1 to 7 through wireless or wired communication and using the communication unit 250 as an input unit.

In addition, biological information to be detected by each of the biological information measuring apparatuses 1 to 7 is not limited to pulse waves (pulse rate), and the biological information measuring apparatus may be an apparatus that detects biological information (for example, oxygen saturation in the blood, body temperature, heartbeat, and the like) other than pulse waves.

### Eighth Embodiment

Next, an eighth embodiment of the invention will be described with reference to the accompanying drawings.

Similarly to the first embodiment described above, the biological information measuring apparatus (hereinafter, referred to as a measuring apparatus) according to the eighth embodiment is a heart rate monitoring apparatus which is worn on a living body (for example, a human body) of which biological information is measured, and which measures biological information such as a pulse (heart rate). Meanwhile, in the following drawings, each component has a size to the extent that the component can be recognized in the drawing, and thus a description may be given by appropriately making a dimension and proportion of each component different from those of an actual component.

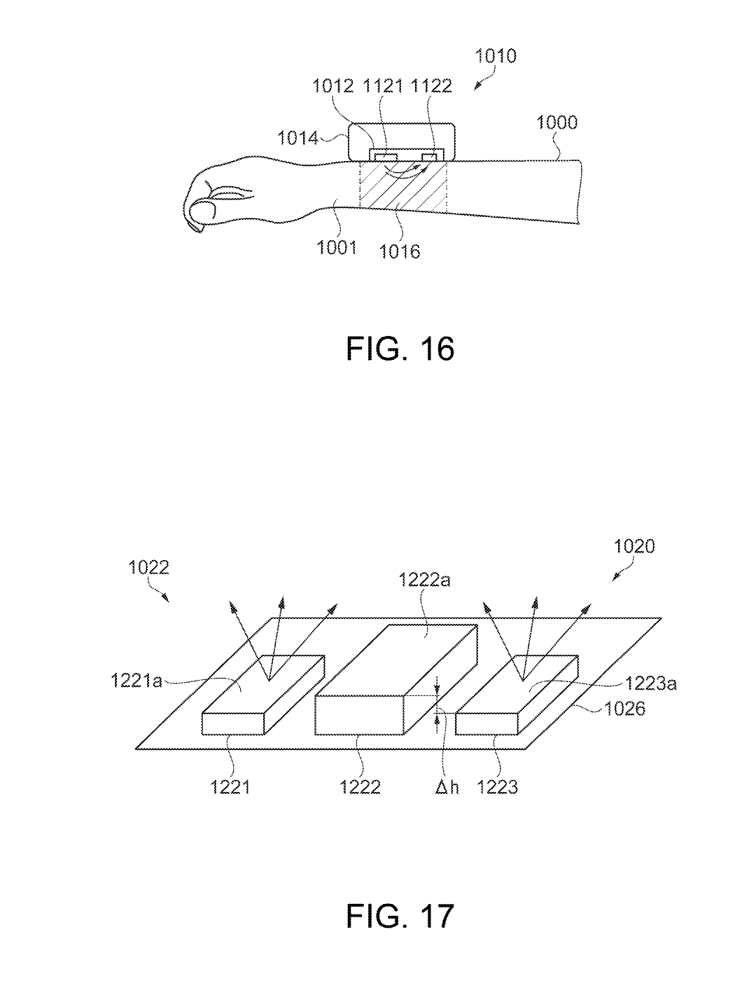

First, before a heart rate monitoring apparatus 1010 as the biological information measuring apparatus according to the eighth embodiment is described, an example of the related art of the heart rate monitoring apparatus as the biological information measuring apparatus according to the eighth embodiment will be described with reference to FIG. 16.

FIG. 16 is a cross-sectional view illustrating a heart rate monitoring apparatus 1010 as a biological information measuring apparatus according to an example of the related art which measures a physiologic parameter (biological information) of a user (test subject) 1000 (the user's arm is shown in the drawing) who is wearing the heart rate monitoring apparatus. The heart rate monitoring apparatus 1010 includes a sensor 1012 that measures a heart rate as at least one physiologic parameter of the user 1000, and a case 1014 that accommodates the sensor 1012. The heart rate monitoring apparatus 1010 is worn on an arm 1001 of the user 1000 by a fixation portion 1016 (for example, a band).

The sensor 1012 is a heart rate monitoring sensor that includes a light emitting element 1121 as a light emitting unit and a light receiving element 1122 as a light receiving unit which are two sensor elements and measures or monitors a heart rate. However, the sensor may be a sensor that measures one or more physiologic parameters (for example, a heart rate, blood pressure, the amount of air inhaled, skin conductivity, skin humidity, and the like). In addition, when the case 1014 includes a band-type housing, the heart rate monitoring apparatus can be used as a wristwatch type monitoring apparatus which is used in, for example, sport. Meanwhile, the case 1014 may have a shape capable of mainly holding the sensor 1012 at a desired position with respect to the user 1000, and may be able to arbitrarily accommodate more elements such as a battery, a processing unit, a display, and a user interface.

The biological information measuring apparatus of the existing example is the heart rate monitoring apparatus 1010 for monitoring a user's heart rate. The sensor 1012 is an optical sensor constituted by the light emitting element 1121 and the light receiving element 1122. An optical heart rate monitor using the optical sensor depends on the light emitting element 1121 (LED is generally used) as a light source that exposes the skin to light. The light emitted from the light emitting element 1121 to the skin is partially absorbed by blood flowing through a blood vessel under the skin, but the rest of the light is reflected and leaves the skin. The reflected light is captured by the light receiving element 1122 (photodiode is generally used). Alight reception signal from the light receiving element 1122 is a signal including information equivalent to the amount of blood flowing through the blood vessel. The amount of blood flowing through the blood vessel varies depending on pulse of the heart. In this manner, a signal on the light receiving element 1122 varies in response to the pulsation of the heart. In other words, a variation in the signal of the light receiving element 1122 is equivalent to the pulse of a heart rate. A pulse rate per unit time is counted (for example, per 10 seconds), to thereby obtain the number of beats of the heart for one minute (that is, a heart rate).

Hereinafter, a heart rate monitoring apparatus 1020 as the biological information measuring apparatus according to the eighth embodiment will be described with reference to FIG. 17. FIG. 17 is a perspective view illustrating a heart rate monitoring apparatus as the biological information measuring apparatus according to the eighth embodiment. Although not shown in FIG. 17, the heart rate monitoring apparatus 1020 as the biological information measuring apparatus according to the eighth embodiment is worn on a user's arm by a fixation portion such as a band, similar to the first embodiment described above.

The heart rate monitoring apparatus 1020 as the biological information measuring apparatus according to the eighth embodiment includes a plurality of light emitting units. In detail, in the heart rate monitoring apparatus 1020, light emitting elements 1221 and 1223 as a plurality of (two in this example) light emitting units and a light receiving element 1222 as one light receiving unit are disposed so as to be lined up in a row. Specifically, a sensor 1022 (in this example, two light emitting elements 1221 and 1223 as a first light emitting unit and a second light emitting unit and the light receiving element 1222 as a light receiving unit are used as three sensor elements) which includes at least two sensor elements is provided.

The light receiving element 1222 as the light receiving unit is disposed between the two light emitting elements 1221 and 1223 as the first light emitting unit and the second light emitting unit. In addition, two light emitting elements 1221 and 1223 as the first light emitting unit and the second light emitting unit are disposed at line symmetrical positions with respect to a virtual line passing through the center of the light receiving element 1222 as the light receiving unit. The light emitting elements 1221 and 1223 and the light receiving element 1222 are disposed in such a manner, and thus it is possible to reduce dead space and to achieve space saving. In addition, light beams from both the first light emitting unit and the second light emitting unit, which are located at line symmetrical positions, gather in the light receiving unit, and thus detection can be performed more accurately.

The sensor element detects a sensor signal. The sensor 1022 includes an optical sensor constituted by the light emitting elements 1221 and 1223 using two LEDs for emitting light to the skin of a user, and at least one light receiving element 1222 (photodiode) for receiving the light reflected from the skin. Further, the heart rate monitoring apparatus 1020 includes a case or a housing (not shown). The case or the housing may be similar to or the same as the case 1014 illustrated in FIG. 16, or may be similar to or the same as the case portion 30 in the first embodiment described above.

The sensor 1022 is carried on one surface of a carrier (substrate) 1026. Here, a configuration including the carrier (substrate) 1026 and the sensor 1022 carried on the carrier (substrate) 1026 corresponds to a biological information measuring module. Meanwhile, the same is true of the ninth to twelfth embodiments. Light emitted from the light emitting elements 1221 and 1223 can be reflected without being absorbed into the skin or the like, and can directly reach the light receiving element 1222. In the heart rate monitoring apparatus 1020, a distance between the carrier 1026 and each of upper surfaces 1221a and 1223a of the respective light emitting elements 1221 and 1223 is smaller than a distance between the carrier 1026 and an upper surface 1222a of the light receiving element 1222. That is, a difference between the distance between the carrier 1026 and each of the upper surfaces 1221a and 1223a of the respective light emitting elements 1221 and 1223 and the distance between the carrier 1026 and an upper surface 1222a of the light receiving element 1222 is Δh. The light receiving element 1222 receives light from the upper surface 1222a thereof which is the uppermost surface layer. According to these configurations, there is an effect that the most of light emitted from the light emitting elements 1221 and 1223 is directed to the skin and reflected light is directly incident on the light receiving element 1222 without going through an air layer or the like. In other words, since a structure in which the light receiving element 1222 comes into close contact with the skin is formed, a structure in which a gap is not likely to be generated between the upper surface (light receiving surface) 1222a of the light receiving element 1222 and the skin can be formed, and thus it is possible to prevent light, such as external light, which serves as a noise source from being incident on the upper surface 1222a. In addition, light from the light emitting elements 1221 and 1223 which does not pass through the skin, for example, light being directly incident on the light receiving element 1222 from the light emitting elements 1221 and 1223 cannot reach the upper surface 1222a of the light receiving element 1222.

### Ninth Embodiment

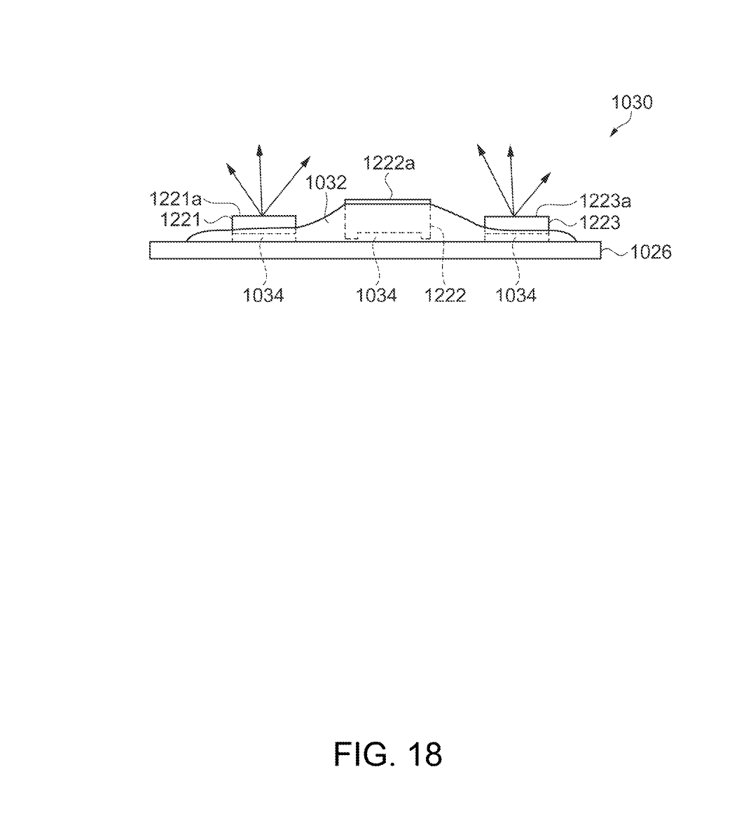

Next, a heart rate monitoring apparatus 1030 as the biological information measuring apparatus according to the ninth embodiment will be described with reference to FIG. 18. FIG. 18 is a side view illustrating a heart rate monitoring apparatus as the biological information measuring apparatus according to the ninth embodiment. Meanwhile, although not shown in FIG. 18, the heart rate monitoring apparatus 1030 as the biological information measuring apparatus according to the ninth embodiment is worn on a user's arm by a fixation portion such as a band, similar to the first embodiment described above.

As illustrated in FIG. 18, electric connection terminals 1034 of light emitting elements 1221 and 1223 as light emitting units and a light receiving element 1222 as a light receiving unit have to be preferably covered with an insulating material (for example, epoxy resin) 1032 in order to protect electrical elements. In addition, a configuration can be adopted in which the insulating material 1032 does not cover the light emitting elements 1221 and 1223 and the light receiving element 1222. Specifically, a configuration can be adopted in which the insulating material 1032 is buried in a region between the light emitting element 1221 and the light receiving element 1222 and a region between the light emitting element 1223 and the light receiving element 1222. In other words, a configuration can be adopted in which at least an upper surface 1222a of the light receiving element 1222 and upper surfaces 1221a and 1223a of the light emitting elements 1221 and 1223 are not covered with the insulating material 1032. With such a configuration, it is possible to suppress disturbance due to an air gap between the skin and the light emitting elements 1221 and 1223. Further, a configuration may be adopted in which the insulating material 1032 covers the upper surfaces 1221a and 1223a of the light emitting elements 1221 and 1223 and the upper surface 1222a of the light receiving element 1222. With such a configuration, the upper surface 1222a of the light receiving element 1222 which comes into contact with the skin and the upper surfaces 1221a and 1223a of the light emitting elements 1221 and 1223 can be protected, and thus it is possible to prevent the upper surface 1222a of the light receiving element 1222 and the upper surfaces 1221a and 1223a of the light emitting elements 1221 and 1223 from being damaged. In this case, the insulating material 1032 can be regarded as a protection film.

In the heart rate monitoring apparatus 1030 as the biological information measuring apparatus according to the ninth embodiment, the insulating material 1032 using an epoxy resin is provided, as an example which is generally implementable. In FIG. 18, the insulating material 1032 is disposed so as not to cover the upper surfaces 1221a and 1223a of the light emitting elements 1221 and 1223, and protects the electric connection terminals 1034. Light beams emitted from the light emitting elements 1221 and 1223 are indicated by an arrow.

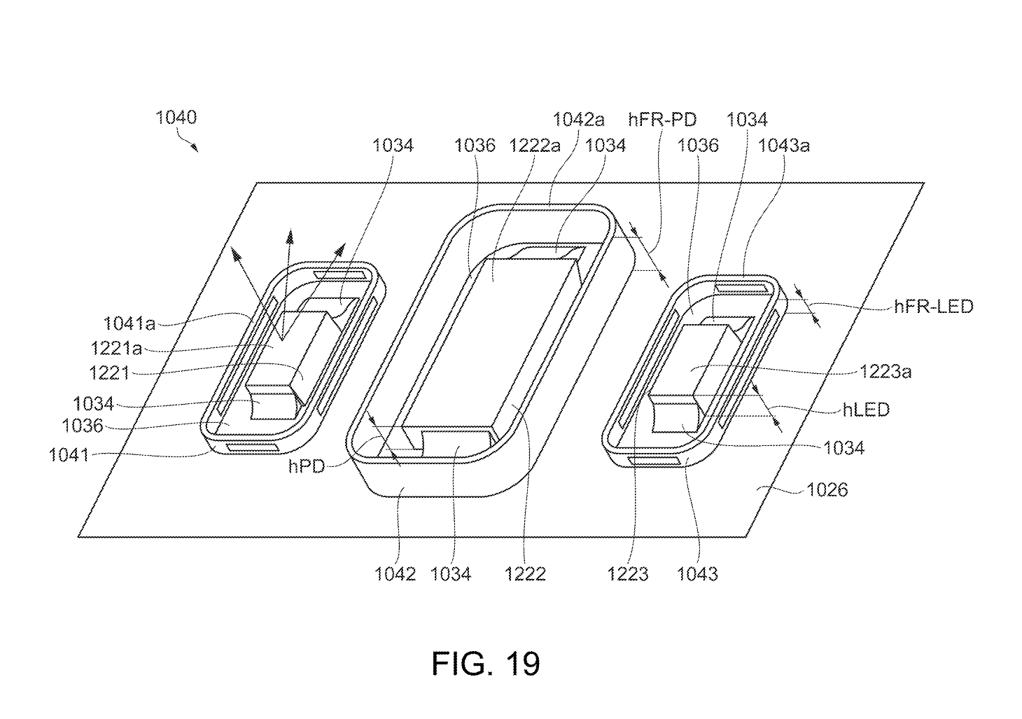

In this manner, the insulating material 1032 is minimally disposed to the extent that a correct function of the heart rate monitoring apparatus 1030 is not hindered, and thus the heart rate monitoring apparatus 1030 can be further improved by protecting the electric connection terminals 1034 of the light emitting elements 1221 and 1223 and the light receiving element 1222. Meanwhile, it is more preferable to configure a heart rate monitoring apparatus 1040 as the biological information measuring apparatus according to the tenth embodiment as illustrated in FIG. 19, instead of adopting the configuration of the ninth embodiment in which an epoxy resin is injected.

### Tenth Embodiment

Next, a heart rate monitoring apparatus 1040 as the biological information measuring apparatus according to the tenth embodiment will be described with reference to FIG. 19. FIG. 19 is a perspective view illustrating a heart rate monitoring apparatus as the biological information measuring apparatus according to the tenth embodiment. Meanwhile, although not shown in FIG. 19, the heart rate monitoring apparatus 1040 as the biological information measuring apparatus according to the tenth embodiment is worn on a user's arm by a fixation portion, such as a band, similar to the first embodiment described above.

In the heart rate monitoring apparatus 1040 as the biological information measuring apparatus according to the tenth embodiment, frames 1041, 1042, and 1043 created are disposed. The frames 1041, 1042, and 1043 are disposed in the vicinity of the light emitting elements 1221 and 1223 as light emitting units and the light receiving element 1222 as a light receiving unit, and a space 1036 is formed between each of the frames 1041, 1042, and 1043 and each of the light emitting elements 1221 and 1223 and the light receiving element 1222. An insulating material (not shown in FIG. 19) is injected with the frames 1041, 1042, and 1043 as guides to cover the electric connection terminals 1034 of the light emitting elements 1221 and 1223 and the light receiving element 1222.

In the example shown in the tenth embodiment, the light emitting elements 1221 and 1223 and the light receiving element 1222 are surrounded by the respective frames 1041, 1042, and 1043. Meanwhile, as another example, all of the frames 1041, 1042, and 1043 may be coupled to each other, or all of the sensor elements may be surrounded by an integrated frame. Meanwhile, the frames 1041, 1042, and 1043 can be used as light shielding walls as examples of light shielding units. The frames 1041, 1042, and 1043 are used as light shielding walls, and thus it is possible to prevent light emitted from the light emitting elements 1221 and 1223 from being directly incident on the light receiving element 1222.

As an improvement for preventing the function of the heart rate monitoring apparatus 1040 from being affected, it is preferable that upper edges 1041a and 1043a of the frames 1041 and 1043 in the vicinity of the light emitting elements 1221 and 1223 are lower than the upper surfaces 1221a and 1223a of the light emitting elements 1221 and 1223. In other words, a distance hFR-LED between the carrier 1026 and each of the upper edges 1041a and 1043a of the respective frames 1041 and 1043 is the same as or smaller than a distance hLED between the carrier 1026 and each of the upper surfaces 1221a and 1223a of the light emitting elements 1221 and 1223 which are surrounded by the respective frames 1041 and 1043 (hFR-LED≦hLED).

It is preferable that a difference between the distance hLED between the carrier 1026 and each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.1 mm to 0.8 mm. Meanwhile, it is more preferable that a difference between the distance hLED between the carrier 1026 of each of the upper surfaces 1221a and 1223a of the respective light emitting elements 1221 and 1223 and the distance hFR-LED between the carrier 1026 and each of the upper edges 1041a and 1043a of the respective frames 1041 and 1043 is set to be in a range from 0.2 mm to 0.5 mm.

In addition, it is preferable that an upper edge 1042a of the frame (receiver frame) 1042 in the vicinity of the light receiving element 1222 is higher than the upper surface 1222a of the light receiving element 1222. In other words, a distance hFR-PD between the carrier 1026 and the upper edge 1042a of the frame 1042 is larger than a distance hPD between the carrier 1026 and the upper surface 1222a of the light receiving element 1222 surrounded by the frame 1042 (hFR-PD>hPD).

It is preferable that a difference between the distance hPD between the carrier 1026 and the upper surface 1222a of the light receiving element 1222 and the distance hFR-PD between the carrier 1026 of the upper edge 1042a of the frame 1042 is set to be in a range from 0 mm to 0.5 mm. Meanwhile, it is more preferable that a difference between the distance hPD between the carrier 1026 and the upper surface 1222a of the light receiving element 1222 and the distance hFR-PD between the carrier 1026 of the upper edge 1042a of the frame 1042 is set to be in a range from 0.1 mm to 0.2 mm.

Further, the distance hFR-PD between the carrier 1026 and the upper edge 1042a of the frame 1042 is larger than the distance hLED between the carrier 1026 and the upper surfaces 1221a and 1223a of the respective light emitting elements 1221 and 1223 (hFR-PD>hLED).

Meanwhile, for example, when the light receiving element 1222 and the light emitting elements 1221 and 1223 are close to each other, a configuration may be adopted in which only one frame wall is present between the light receiving element 1222 and each of the light emitting elements 1221 and 1223. This may occur because of manufacturing easiness. When the one frame wall is a case, frame walls of the frames of both the light receiving element 1222 and each of the light emitting elements 1221 and 1223 are coincident with each other. This means that the frame walls of the light emitting elements 1221 and 1223 become relatively high. In detail, the frame wall on the light receiving element 1222 side out of the frame walls of the frames 1041 and 1043 surrounding the respective light emitting elements 1221 and 1223 becomes relatively high, and the other frame wall becomes lower than the upper surfaces 1221a and 1223a of the respective light emitting elements 1221 and 1223.

Further, instead of the frames 1041, 1042, and 1043, a configuration may be adopted in which a first wall portion is provided between the light receiving element 1222 and the light emitting element 1221 or the light emitting element 1223 and a second wall portion is provided on the outside of the light emitting elements 1221 and 1223, that is, on the side opposite to the first wall portion with respect to the light receiving element 1222.

In such a configuration, a distance between the carrier 1026 and the upper surface of the first wall portion may be larger than a distance between the carrier 1026 and the upper surface of the second wall portion. With such a configuration, it is possible to realize the function of the frame using a smaller number of members than in a case where a light emitting element and a light receiving element are surrounded as illustrated in FIG. 19.

Meanwhile, the frames 1041 and 1043 and the frame 1042 are used as in the tenth embodiment, and thus it is possible to prevent an insulating material to be injected, such as an epoxy resin, from flowing out. In this manner, the partitioning of an insulating material such as an epoxy resin by creating an additional structure is option of allowing high mass productivity to be obtained. Meanwhile, the frames 1041 and 1043 and the frame 1042 may be formed of the same material as that of the carrier 1026. For example, the frames may be formed by injection molding using an epoxy-based resin or a polycarbonate-based resin.

As described above, the insulating material 1032 (see FIG. 18) protects the electric connection terminals 1034 of the sensor elements (light emitting elements 1221 and 1223 and the light receiving element 1222). However, the electric connection terminals 1034 have to further come into contact with additional electronic apparatuses (for example, a driver, detection electronics, a processor, or a power supply) which are other elements. This means that there is any electrical connection between the carrier 1026 (may be a printed circuit board (PCB)) and the additional electronic apparatuses. In addition, the structure of the heart rate monitoring apparatus according to this embodiment can be applied not only to an apparatus for measuring a heart rate but also to apparatuses for measuring pulse waves and pulse.

### Eleventh Embodiment

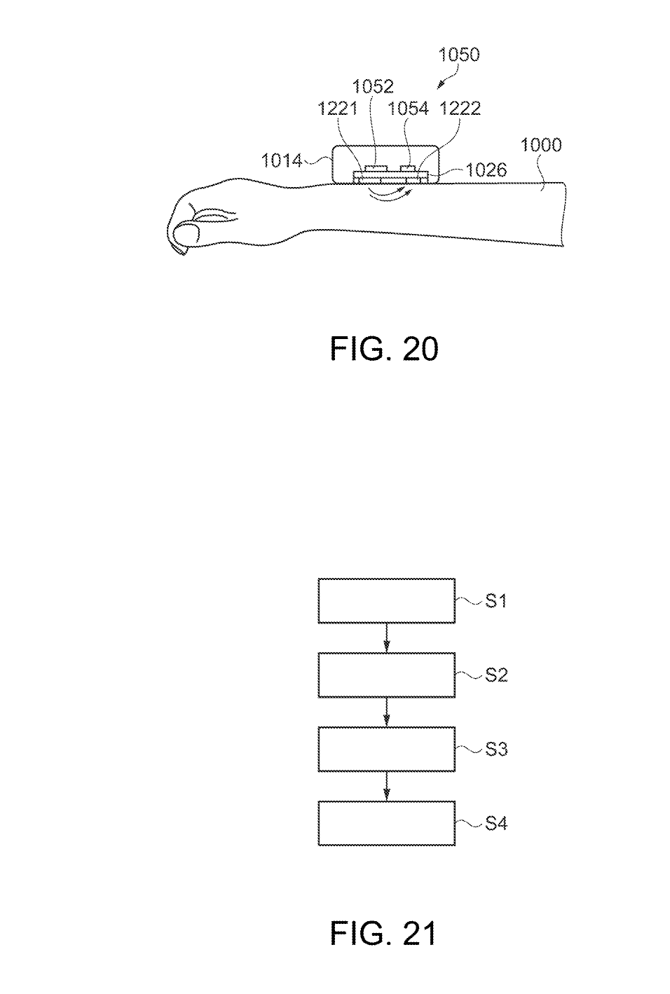

A heart rate monitoring apparatus 1050 as the biological information measuring apparatus according to the eleventh embodiment will be described with reference to FIG. 20. FIG. 20 is a cross-sectional view illustrating a heart rate monitoring apparatus as the biological information measuring apparatus according to the eleventh embodiment. Meanwhile, although not shown in FIG. 20, the heart rate monitoring apparatus 1050 as the biological information measuring apparatus according to the eleventh embodiment is worn on a user's arm by a fixation portion such as a band, similar to the first embodiment described above.

The heart rate monitoring apparatus 1050 as the biological information measuring apparatus according to the eleventh embodiment includes the above-mentioned additional electronic apparatuses (for example, a processor 1052 and a driver 1054). An external electric connection terminal (not shown) is not disposed on a carrier 1026 which is the same as that on which sensor elements (light emitting element 1221 as a light emitting unit and a light receiving element 1222 as a light receiving unit) are disposed. In other words, the additional electronic apparatuses are disposed on a carrier different from the carrier on which the sensor elements are disposed, or a substrate. With such a configuration, it is possible to mount necessary additional electronic apparatuses on the heart rate monitoring apparatus 1050 while maintaining a satisfactory contact between the skin and the sensor elements (light emitting element 1221 and the light receiving element 1222). For example, the external electric connection terminal can be disposed on the side surface of the carrier 1026.

As described above, different types of sensors can be used in the biological information measuring apparatus according to the invention. For example, when the light receiving element 1222 mentioned above is an electric sensor, two skin conductance electrodes (for example, sensor elements (the light emitting element 1221 and the light receiving element 1222 which are illustrated in FIG. 20)) which come into contact with the skin of a user and measure the conductivity of the user are covered with the skin. Meanwhile, two or more types of sensors can be used in such a type of biological information measuring apparatus, and the number of sensor elements does not matter.

In the eighth to eleventh embodiments, a flow chart of a method of manufacturing the proposed biological information measuring apparatus that measures a physiologic parameter is illustrated in FIG. 21.

In first step S1, the sensor 1022 including at least two sensor elements (the light emitting element 1221 and the light receiving element 1222) for detecting a sensor signal is disposed on the carrier 1026. In second step S2, an electrical contact between the sensor elements is formed in the carrier 1026. In third step S3, one or more frames 1041 and 1042 is formed on the carrier 1026 in the vicinity of the sensor 1022 and/or the individual sensor elements (the light emitting element 1221 and the light receiving element 1222). In fourth step S4, the insulating material 1032 is injected into and filled in regions surrounded by the respective frames 1041 and 1042 so as not to cover the upper surfaces 1221a and 1222a of the sensor elements (the light emitting element 1221 and the light receiving element 1222) which are provided on the carrier 1026.

According to the eighth to eleventh embodiments described above, a method of protecting an electrical contact that does not exert a bad influence on the performance of the biological information measuring apparatus is proposed. The biological information measuring apparatus is formed by such a method as that in which the performance of a sensor is maintained. For example, at least one of the frames 1041 and 1043 prevents the position of the sensor with respect to the skin from being shifted. Further, at least one of the frames 1041 and 1043 can help emitted direct light to be prevented from being input to the light receiving element 1222. It is preferable that the heights of the frames 1041 and 1043, facing the light receiving element 1222, in the vicinity of the respective light emitting elements 1221 and 1223 have to be smaller than the heights of the upper surfaces 1221a and 1223a of the respective light emitting elements 1221 and 1223. In addition, the frame 1042 in the vicinity of the light receiving element 1222 may be higher than the upper surface 1222a of the light receiving element 1222.

Also in the biological information measuring apparatuses according to the eighth to eleventh embodiments, it is possible to increase an operable time of the biological information measuring apparatus capable of measuring biological information with a high level of accuracy by applying an intermittent operation.

### Twelfth Embodiment

The biological information measuring apparatuses of the first to eleventh embodiments described above may include various types of sensors such as, a strain gauge, a thermometer, a clinical thermometer, an acceleration sensor, a gyro sensor, a piezoelectric sensor, a pressure sensor, a sphygmomanometer, an electrochemical sensor, a global positioning system (GPS), and a vibrometer. The biological information measuring apparatuses include these sensors, and thus it is possible to derive information regarding a personal physiological state on the basis of data indicating one or one or more physiological parameters, such as heartbeat, pulse, a variation between pulsations, an elektrokardiogram (EKG), an electrocardiogram (ECG), a respiration rate, a skin temperature, a body temperature, a body heat flow, a galvanic skin response, a galvanic skin reflex (GSR), an electromyogram (EMG), an electroencephalogram (EEG), an electrooculography (EOG), blood pressure, body fat, a hydration level, an activity level, a body motion, oxygen consumption, glucose, a blood glucose level, muscle mass, pressure applied to a muscle, pressure applied to a bone, ultraviolet absorption, a sleep state, a physical condition, a stress state, and a posture (for example, lying, standing upright, and sitting). In addition, values obtained by the various types of sensors are transmitted to, for example, a portable communication terminal such as a smartphone, a mobile phone, or a feature phone, or an information processing terminal such as a computer or a tablet computer, so that the portable communication terminal or the information processing terminal may execute the arithmetic processing of the physiological parameters.

A user inputs his or her own profile to the biological information measuring apparatus, the portable communication terminal, or the information processing terminal before measuring biological information. Thereby, the user can receive user's unique characteristic information and environmental information which are required to be coped with, in order to maximize a possibility of a recommended healthy lifestyle being established and maintained, on the basis of the profile and biological information measurement results. Examples of information to be provided include one or two or more of exercise information such as an exercise type, an exercise strength, and an exercise time, meal information such as a meal time, the amount of meal, recommended intake ingredients and intake menus, and intake ingredients and intake menus that should be avoided, life support information such as a sleep time, the depth of sleep, the quality of sleep, a wake-up time, a landing time, a working time, stress information, consumed calories, intake calories, and calorie balance, physical information such as basal metabolism, the amount of body fat, a body fat percentage, and muscle mass, medication information, supplement intake information, and medical information.

Examples of the user's own profile input in advance include one or two or more of the age, the date of birth, the sex, hobbies, an occupation type, a blood type, a past sports history, an activity level, meal, the regularity of sleep, the regularity of bowel habit, situation adaptability, durability, responsiveness, the strength of reaction, user's personality such as a temper, a user's self-independence level, independent formation, self-management, sociability, a memory and an academic attainment ability, a user's awakening level, a perception speed, an ability to avoid attention alienation factors, user's attention including an awakening state and a self-supervision ability, an attention continuance ability, the weight, the height, blood pressure, a user's health state, medical examination results by a doctor, the date of a medical examination by a doctor, the presence or absence of a contact between a doctor and a health care person, medicines and supplements that are currently taken, the presence or absence of an allergy, an allergy history, the current allergy symptoms, an opinion of behavior pertaining to health, a user's disease history, a user's operation history, a family medical history, a social phenomenon, such as a divorce or unemployment, which is required to be adjusted by an individual, conviction pertaining to a user's health priority, a sense of values, an ability to change behavior, a phenomenon considered to be a cause of the stress of life, a stress management method, the degree of user's own consciousness, the degree of user's empathy, the degree of user's authority transfer, user's pride, user's exercise, a sleep state, a relaxed state, the current routine of daily activity, the personality of an important person (for example, a spouse, a friend, a colleague, or a superior officer) in the user's life, and a user's way to catch whether a conflict that disturbs a healthy lifestyle or contributes to stress is present in a relationship with an important person.

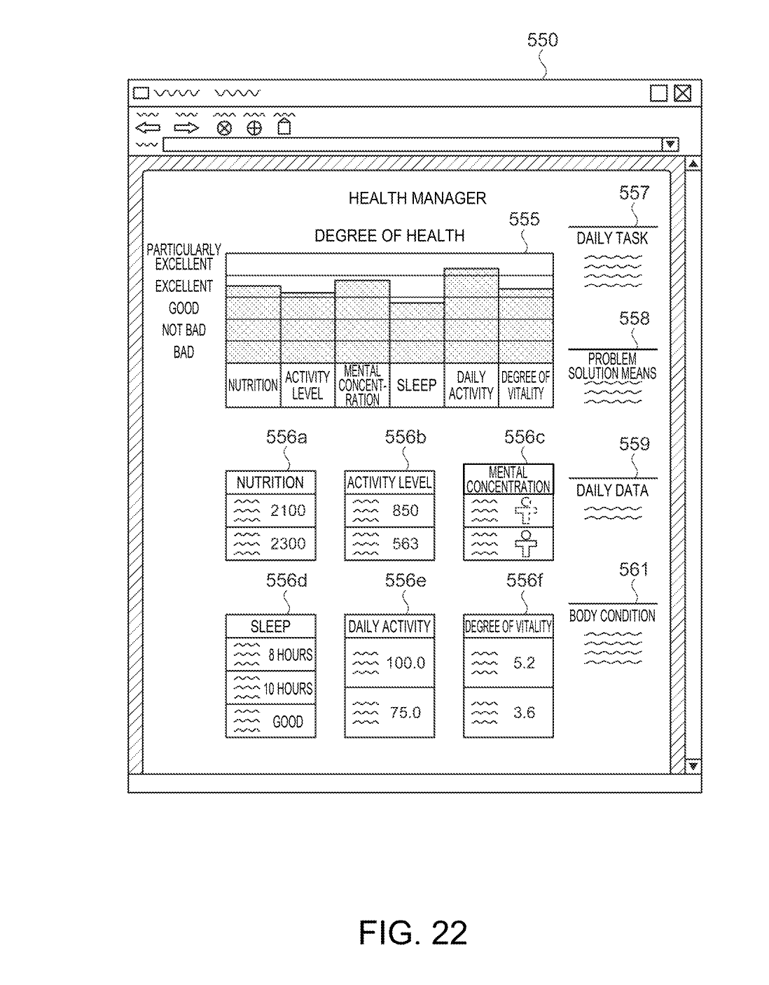

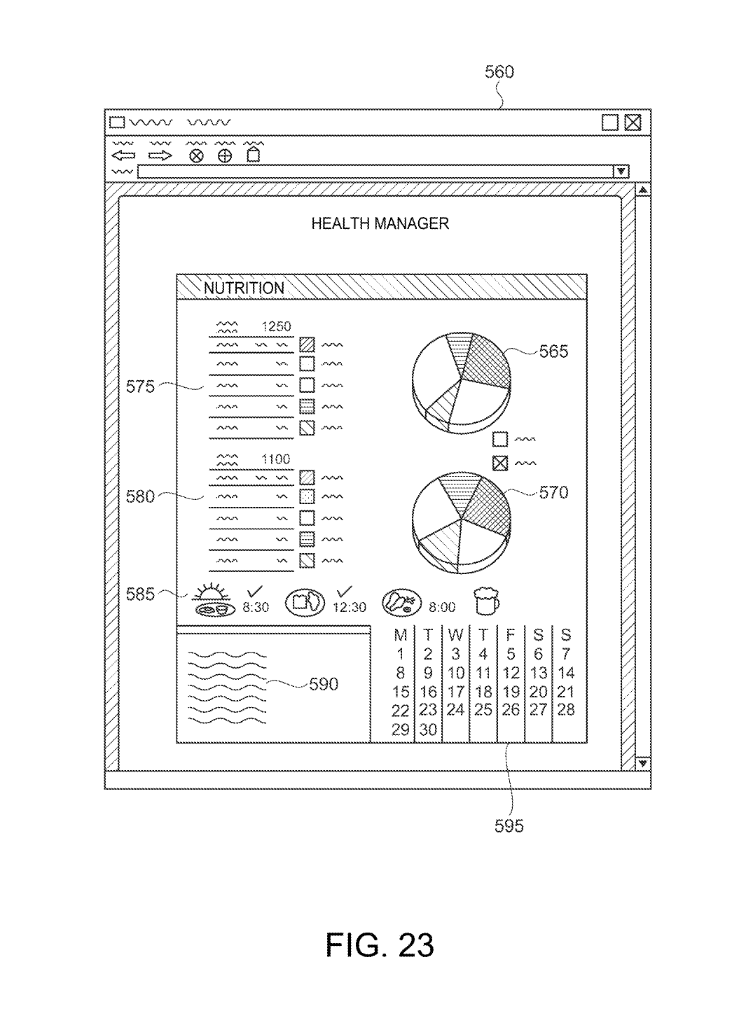

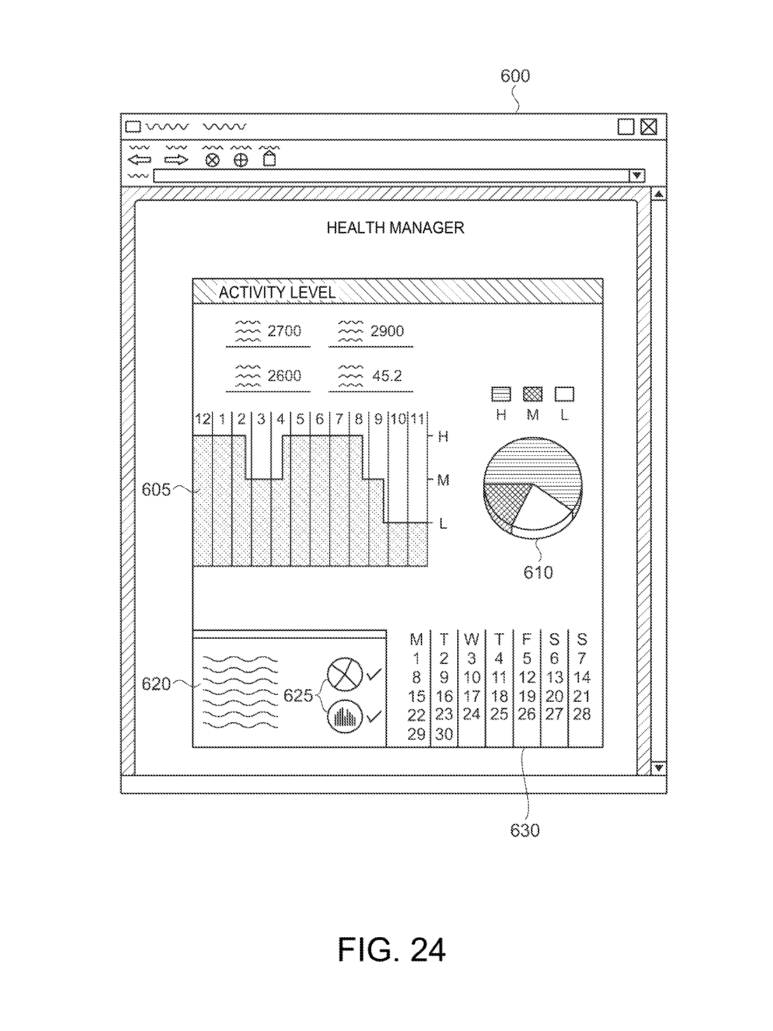

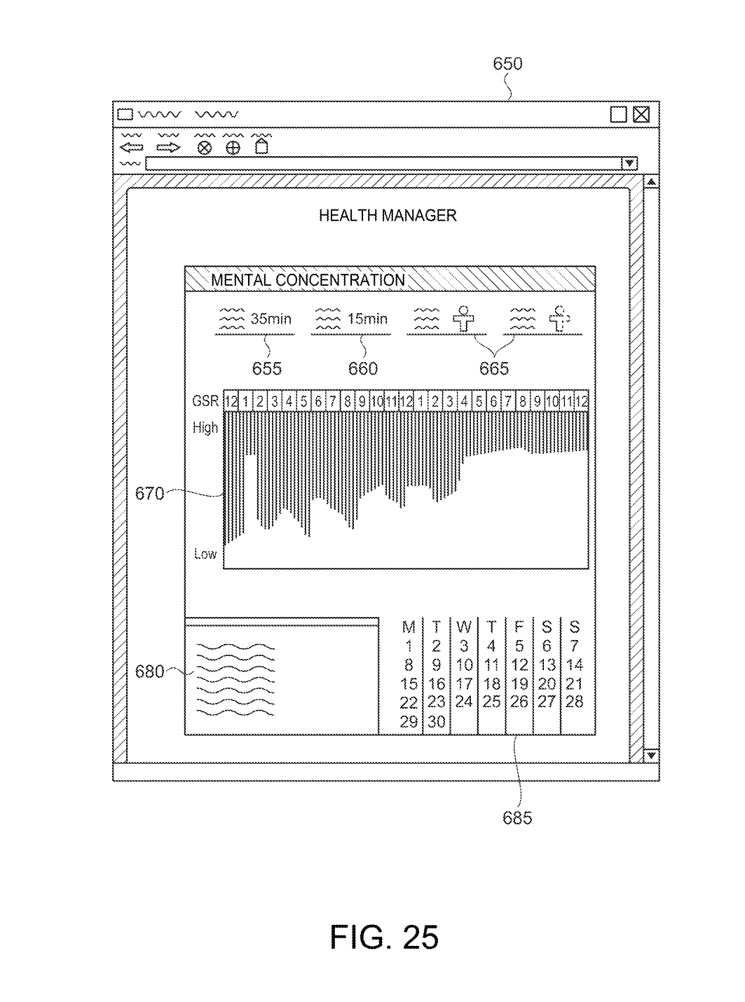

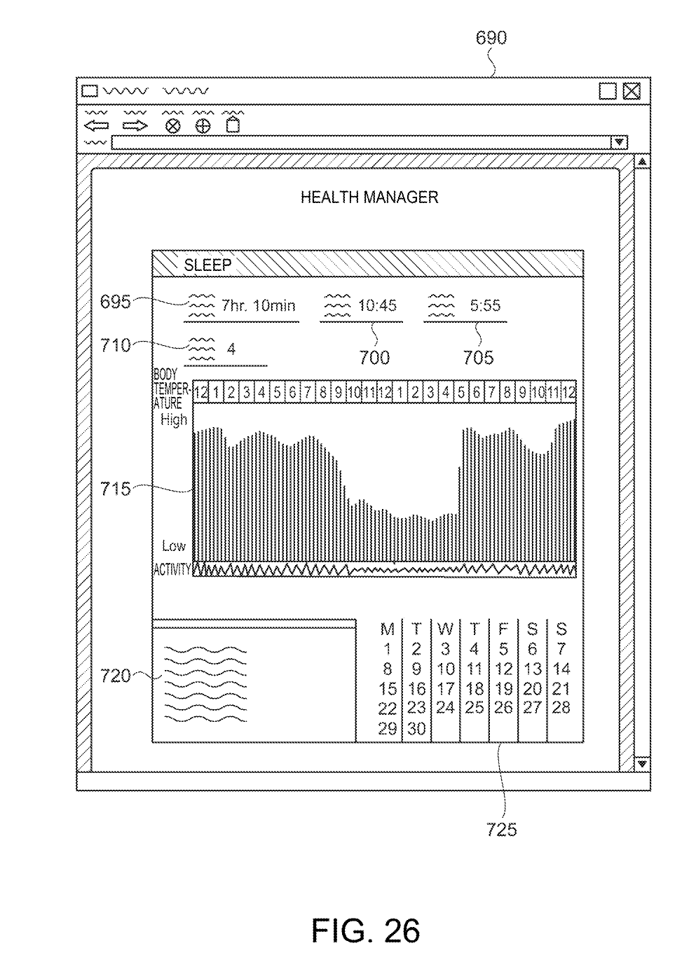

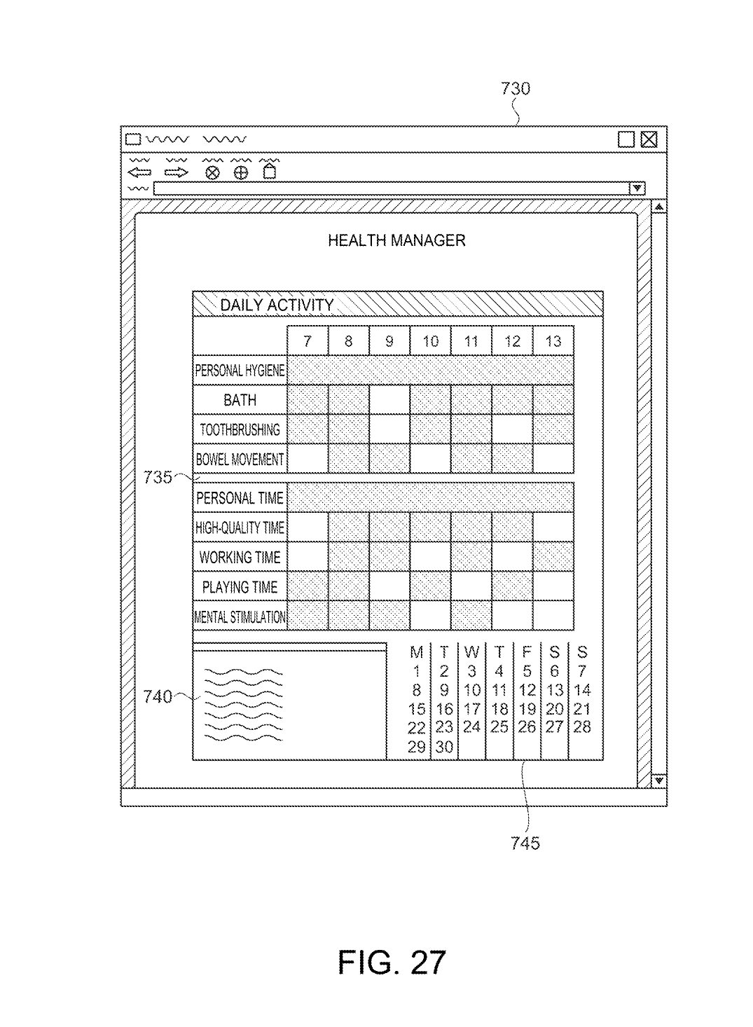

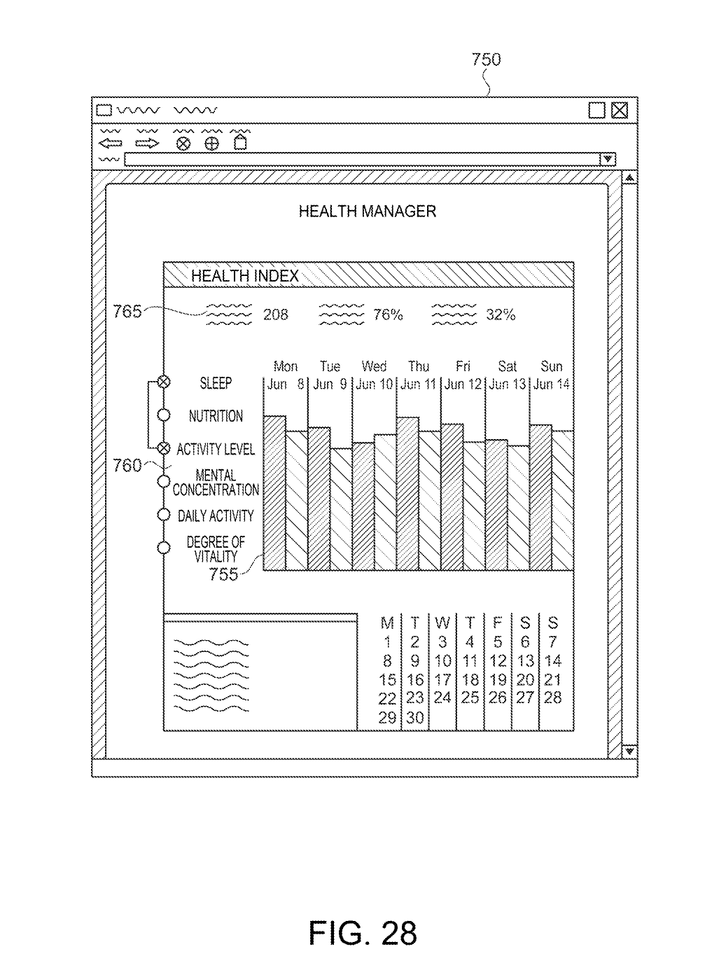

Here, reference will be made to FIGS. 22 to 28 to describe a biological information measuring apparatus according to a twelfth embodiment which is capable of receiving user's unique characteristic information and environmental information which are required to be coped with, in order to maximize a possibility of a recommended healthy lifestyle being established and maintained. FIG. 22 is a schematic diagram illustrating a web page serving as a starting point of a health manager in the biological information measuring apparatus of the twelfth embodiment. FIG. 23 is a diagram illustrating an example of a nutrition web page, and FIG. 24 is a diagram illustrating an example of an activity level web page. In addition, FIG. 25 is a diagram illustrating an example of a mental concentration web page, and FIG. 26 is a diagram illustrating an example of a sleep web page. In addition, FIG. 27 is a diagram illustrating an example of a daily activity web page, and FIG. 28 is a diagram illustrating an example of a vitality degree web page.

Although not shown in the drawing, the biological information measuring apparatus according to the twelfth embodiment includes, for example, a sensor device which is connected to a microprocessor. In the biological information measuring apparatus according to the twelfth embodiment, pieces of data regarding various life activity items which are finally transmitted to a monitor unit and stored, and personal data or living information which is input by a user from a website maintained by the monitor unit are processed by the microprocessor and are provided as biological information. Hereinafter, a specific example will be described.

A user has access to a health manager for the user through a web page, application software, and other communication media. FIG. 22 illustrates a web page 550 serving as a starting point of the health manager, as an example. In the web page 550 of the health manager shown in FIG. 22, various pieces of data are provided to a user. The provided data is one or more pieces of data of, for example, (1) data indicating various physiological parameters based on values measured by various sensor devices, (2) data derived from data indicating various physiological parameters, and (3) data indicating various context parameters generated by the sensor device and data input by the user.

Analysis state data has features that a certain utility or algorithm is used in order to perform conversion into (1) data indicating various physiological parameters acquired by the sensor device, (2) data derived from various physiological parameters, (3) the degree of health obtained by calculating one or more pieces of data of data indicating various context parameters acquired by the sensor device and data input by the user, (4) the degree of good health and a lifestyle index, and the like. For example, it is possible to calculate the amounts of calories, protein, fat, carbohydrates, and certain vitamin on the basis of data input by the user in relation to food taken. In addition, as another example, it is possible to provide indexes of stress levels over a desired period of time to the user by using a skin temperature, a heart rate, a respiration rate, a heat flow and/or a GSR. As still another example, it is possible to provide indexes of sleep patterns over a desired period of time to the user by using a skin temperature, a heat flow, a variation between pulsations, a heart rate, a pulse rate, a respiration rate, a central body temperature, a galvanic skin response, an EMG, an EEG, an EOG, blood pressure, oxygen consumption, ambient sounds, and body motion detected by a device such as an accelerometer.

In the web page 550 illustrated in FIG. 22, a health index 555 as the degree of health is displayed. The health index 555 is a graphic utility for measuring the degree of achievement of user's results and a recommended healthy daily task and giving feedback to member users. In this manner, the health index 555 indicates health states and progress conditions of action pertaining to health maintenance of the member users. The health index 555 includes six categories regarding the health and lifestyle of a user, that is, nutrition, an activity level, mental concentration, sleep, daily activity, and the degree of vitality (overall impression). The category of “nutrition” pertains to information regarding what, when, and how much the person (user) has eat and taken. The category of “activity level” pertains to the amount of exercise regarding how much the person has moved around. The category of “mental concentration” pertains to the quality (ability) of the activity for making the person (user) set to be in a relaxed state in a state where the mind of the person is in a highly concentrated state, and to a period of time for which the person concentrates on the activity. The category of “sleep” pertains to the quality and amount of sleep of the person (user). The category of “daily activity” pertains to matters that have to be performed every day by the person (user) and to health risks that the person meets with. The category of “the degree of vitality (impression)” pertains to a general way to catch whether being in a good mood on a certain day. Preferably, each of the categories includes a level display or a bar graph indicating how many results the user has attained on a scale varying between “bad” and “good”.

When each member user terminates the above-mentioned initial examination, a profile for providing user's own characteristics and a summary of a living environment to the user is created, and recommended healthy daily tasks and/or targets are presented. The recommended healthy daily tasks include any combination in specific pieces of advices regarding appropriate nutrition, exercise, mental concentration, and user's daily activity (life). A model schedule or the like may be presented as a guide indicating how to take activity items pertaining to the recommended healthy daily tasks in the user's life. The user is regularly subjected to the examination, and practices the above-mentioned items accordingly on the basis of the results thereof.

The category of “nutrition” is calculated from both data input by a user and data sensed by a sensor device. The data input by the user includes the times for breakfast, lunch, and dinner, and any snack and the eating and drinking times thereof, and food to be eaten and drunk, supplements such as vitamin, and water or another liquid (drinking water or liquid food) which is drunk during a time which is selected in advance. A central monitoring unit calculates consumed calories or well-known nutritional values such as the contents of protein, fat, carbohydrates, vitamin, and the like, on the basis of the data and stored data regarding known characteristics of various articles of food.

In the category of “nutrition”, a recommended healthy daily task can be determined on the basis of the bar graph indicating the nutrition of the health index 555. The recommended healthy daily task can be adjusted on the basis of information such as the sex, age, and height/weight of a user. Meanwhile, a user or a representative of the user can set a target of certain nutrition pertaining to the amount of calories consumed every day, the amount of nutriments such as protein, fiber, fat, and carbohydrates, the amount of water, and ratios thereof to the total intake. Parameters used for the calculation of the bar graph include the number of meals for one day, the amount of water consumed, and the type and amount of food eaten every day which are input by a user.

Nutritional information is presented to a user by a nutrition web page 560 as illustrated in FIG. 23. It is preferable that the nutrition web page 560 includes nutrition numerical charts 565 and 570 that are pie charts showing actual and target numerical values of nutrition, and nutrition intake charts 575 and 580 showing an actual total nutrition intake amount and a target total nutrition intake amount. In the nutrition numerical charts 565 and 570, it is preferable that items such as carbohydrates, protein, and fat are expressed by percentage. In the nutrition intake charts 575 and 580, it is preferable that a total value and a target value of calories are expressed by being divided into ingredients such as fat, carbohydrates, protein, and vitamin. The nutrition web page 560 includes a history 585 indicating the times when food and water are consumed, a hyperlink 590 that allows a user to be able to directly check a news story pertaining to nutrition, advice for improving a daily task pertaining to nutrition, and any related advertisement on a network, and a calendar 595 in which an application period and the like can be selected. Items indicated by the hyperlink 590 can be selected on the basis of information learned from an individual through examination, and the individual's results measured by the health index.

The category of “activity level” in the health index 555 is designed so as to support a user's check regarding when and how the user was active (moved) on that day, and the like, and both data input by the user and data sensed by the sensor device are used. The data input by the user includes details pertaining to the user's daily activity such as, for example, doing work at the desk from 8 a.m. to 5 p.m. and taking an aerobic lesson from 6 p.m. to 7 p.m. The related data sensed by the sensor device includes a heart rate, an exercise sensed by a device such as an accelerometer, a heat flow, a respiration rate, the amount of calories consumed, a GSR, and a water supply level, and these can be taken out by the sensor device or the central monitoring unit. The amount of calories consumed can be calculated by various methods such as multiplication of the type of exercise which is input by the user and the duration of exercise which is input by the user, multiplication of the sensed exercise, an exercise time, and a filter constant, or multiplication of the sensed heat flow, the time, and a filter constant.

In the category of “activity level”, a recommended healthy daily task can be determined on the basis of the bar graph indicating the activity level of the health index 555. The recommended healthy daily task includes a minimum target calories consumed by the activity, and the like. Meanwhile, the minimum target calories can be set on the basis of information such as the sex, age, height, and weight of a user. Parameters used for the calculation of the bar graph include a time input by the user and/or a time sensed by the sensor device which are times spent for various types of exercises or an energetic lifestyle activity, and the amount of calories burned over an energy consumption parameter which is calculated in advance.

Information regarding the activity (movement) of an individual user is presented to the user by an activity level web page 600 illustrated in FIG. 24. The activity level web page 600 includes an activity degree graph 605, having a bar graph shape, which shows the user's activity monitored according to three categories, that is, “high”, “medium”, and “low” that are classified with respect to a predetermined unit time. An activity percentage chart 610 having a pie chart shape can be presented in order to express a percentage for a predetermined period of time such as, for example, one day which is spent in each of the categories by the user. In addition, the activity level web page 600 may include a calorie display (not shown) for displaying items such as a total amount of calories burned, a target value of daily burned calories, a total value of calories taken, and an aerobic exercise time. The activity level web page 600 includes at least one hyperlink 620 in order to allow the user to be able to directly check a related news story, advice for improving a daily task pertaining to an activity level, and a related advertisement on a network.

The activity level web page 600 can be viewed in various formats, and can be configured such that a user can select a bar graph, a pie chart, or both the graph and the chart and the selection can be performed by an activity level check box 625. An activity level calendar 630 is provided so that an application period and the like can be selected. Items indicated by the hyperlink 620 can be selected on the basis of information extracted from an individual through examination, and the results measured by the health index.

The category of “mental concentration” in the health index 555 is designed so as to support a user's monitoring of parameters pertaining to a time when the activity for allowing the user's body to reach a deep relaxed state while concentrating his or her mind is performed, and is based on both data input by the user and data sensed by the sensor device. In detail, the user can input a starting time and a termination time of a relaxation activity such as yoga or meditation. The quality of these activity items determined by the depth of mental concentration can be measured by monitoring parameters including a skin temperature, a heart rate, a respiration rate, and a heat flow which are sensed by the sensor device. It is also possible to use a variation in the percentage of a GSR obtained by either of the sensor device or the central monitoring unit.

In the category of “mental concentration”, a recommended healthy daily task can be determined on the basis of the bar graph indicating the activity level of the mental concentration in the health index 555. The recommended healthy daily task is displayed inclusive of daily joining in the activity of deeply relaxing a body while making mind set to be in a highly concentrated state. Parameters used for the calculation of the bar graph include the length of time spent for the mental concentration activity, the depth of the mental concentration activity, or a variation in the percentage of a skin temperature, a heart rate, a respiration rate, a heat flow, or a GSR which is sensed by the sensor device from a base line indicating quality.

Information regarding time spent for an action of deeply looking back oneself (introspection) and for mental concentration activity such as deep relaxation of a body is presented to a user by a mental concentration web page 650 illustrated in FIG. 25. Meanwhile, the mental concentration activity may be referred to as a session. The mental concentration web page 650 includes a time 655 spent for the session, a target time 660, comparison portions 665 indicating a target value of the depth of mental concentration and an actual value, and a histogram 670 indicating the overall stress level which is derived from a skin temperature, a heart rate, a respiration rate, a heat flow, and/or a GSR.

In the comparison portion 665, the contour of a human indicating a target mental concentration state is shown by a solid line, and the contour of a human indicating an actual mental concentration state varies between a blurred state (shown by a dashed line in FIG. 25) and a solid line in accordance with the level of mental concentration. In addition, the preferable mental concentration web page 650 includes a hyperlink 680 that allows a user to be able to directly check a related news story, advice for improving a daily task pertaining to mental concentration, and a related advertisement on a network, and a calendar 685 in which an application period can be selected. Items indicated by the hyperlink 680 can be selected on the basis of results measured by information learned from an individual through examination, and the results measured by the health index.

The category of “sleep” in the health index 555 is designed so as to be able to support a user's monitoring of a sleep pattern and the quality of sleep. This category is intended to help a user to learn the importance of sleep in a healthy lifestyle and the relation of sleep to a daily cycle which is an ordinary daily variation in the function of the body. The category of “sleep” is based on both data input by the user and data sensed by the sensor device. The data input by the user between related time intervals includes ranks of a sleep-onset time and a wake-up time (sleep time) of the user and the quality of sleep. The related data obtained by the sensor device includes a skin temperature (body temperature), a heat flow, a variation between pulsations, a heart rate, a pulse rate, a respiration rate, a central body temperature, a galvanic skin response, an EMG, an EEG, an EOG, blood pressure, and oxygen consumption. In addition, ambient sounds and body motion which is detected by a device such as an accelerometer also have relevance. Thereafter, a sleep-onset time, a wake-up time, the interruption of sleep, the quality of sleep, the depth of sleep, and the like can be calculated and derived using the data.

The bar graph showing the sleep in the health index 555 displays a healthy daily task including the securing of a preferable nightly minimum sleep time, a predictable bedtime, and a wake-up time. Specific parameters enabling the calculation of the bar graph include a daily sleep time and a wake-up time which are sensed by the sensor device or input by the user, and the quality of sleep which is graded by the user or derived from another data.

Information regarding the sleep is presented to a user by a sleep web page 690 illustrated in FIG. 26. The sleep web page 690 includes a sleep time display 695 based on either of data from the sensor device or data input by the user, a user bedtime display 700, and a wake-up time display 705. Meanwhile, the quality of sleep which is input by the user can be displayed using a sleep quality rank 710. In addition, when a display exceeding a time interval for one day is performed in the sleep web page 690, the sleep time display 695 can be displayed as a cumulative value, and the bedtime display 700, the wake-up time display 705, and the sleep quality rank 710 can be calculated and displayed as average values. In addition, the sleep web page 690 also includes a sleep graph 715 selectable by a user who calculates and displays one sleep-related parameter during a predetermined time interval. FIG. 26 illustrates a variation in a heat flow (body temperature) for one day. The heat flow tends to be reduced while asleep and to be increased while awake. It is possible to obtain a biorhythm of the person from the information.

In addition, the sleep graph 715 displays data from an accelerometer embedded in the sensor device that monitors body motion. In addition, the sleep web page 690 can include a hyperlink 720 that allows a user to be able to directly check a news story pertaining to sleep, advice for improving a daily task pertaining to sleep, and a related advertisement on a network, and a sleep calendar 725 for selecting a related time interval. Items indicated by the hyperlink 720 can be particularly selected on the basis of information learned from an individual in examination, and results measured by the health index.

The category of “daily activity” in the health index 555 is designed so as to be able to support a user's monitoring of a certain activity, pertaining to health or safety, and risk, and is completely based on data input by a user. The category of “daily activity” which pertains to activity in a daily life includes four categories which are subordinate concepts. Specifically, the category is classified into (1) an item pertaining to personal hygiene which enables a user's monitoring of dental care using a toothbrush or floss or activity such as taking a shower, (2) an item pertaining to health maintenance which enables tracing of whether a user is taking medicine or a supplement as prescribed, and enables a user's monitoring of the consumption of cigarettes or alcohol, and the like, (3) an item pertaining to personal time which enables a user's monitoring of time or leisure, which is spent with the user's family or friend, and mental concentration activity, and (4) an item pertaining to responsibility which enables a user's monitoring of work, such as household chores, and household activity.

In the category of “daily activity”, it is preferable that the bar graph indicating the “daily activity” in the health index 555 displays the following recommended healthy daily tasks. As an example of a daily task pertaining to the personal hygiene, it is preferable that a user takes a shower or takes a bath every day, keeps his or her teeth clean by using a toothbrush or floss every day, and has regular bowel movements. In addition, as an example of a daily task pertaining to the health maintenance, it is preferable that a user takes medicine, vitamin pills, and/or supplements, does not smoke, drinks in moderation, and monitors his or her health every day by a health manager. As an example of a daily task pertaining to the personal time, it is preferable that a user makes at least predetermined time every day in order to spend the time with his or her family, and/or spends high-quality time with his or her friend, reduces time for work, takes time for leisure or play, and performs activity using his or her brain. As an example of a daily task pertaining to the responsibility, it is preferable that a user does household chores, is not late for work, and keeps a promise. The bar graph is determined by information input by a user, and/or is calculated on the basis of the degree to which the user completes activity listed up every day.

Pieces of information regarding these activity items are presented to a user by a daily activity web page 730 illustrated in FIG. 27. An activity chart 735 in the daily activity web page 730 shows whether a user has executed necessary activity by the daily task. In the activity chart 735, one or more of the subordinate concepts can be selected. In the activity chart 735, a box which is colored or shaded indicates that a user has executed necessary activity, and a box which is not colored or shaded indicates that the user has not executed the activity. The activity chart 735 can be created at a selectable time interval and can be viewed. FIG. 27 illustrates the categories of personal hygiene and personal time in a specific week as an example. Further, the daily activity web page 730 may include a hyperlink 740 that allows a user to be able to directly check a related news story, advice for improving a daily task pertaining to activity in a daily life, and a related advertisement on a network, and a daily activity calendar 745 for selecting a related time interval. Items indicated by the hyperlink 740 can be selected on the basis of information learned from an individual in examination, and results determined by the health index.

The category of “the degree of vitality” in the health index 555 is designed so as to enable a user's monitoring of recognition of whether being in good spirits on a specific day, and is based on essentially subjective grade information which is directly input by the user. The user performs ranking using scales of, preferably, 1 to 5 with respect to the following nine areas, that is, (1) mental keenness, (2) the degree of mental and psychological happiness, (3) an energy level, (4) a capacity for stresses of life, (5) the degree of being concerned about appearances, (6) the degree of physical happiness, (7) self-control, (8) a motive, and (9) comfort by a relationship with others. These degrees (grades) are averaged to be used for the calculation of the bar graph of the health index 555.

FIG. 28 illustrates a vitality degree web page 750. The vitality degree web page 750 allows a user to be able to check the degree of vitality during a time interval, selectable by the user, which includes continuous or discontinuous arbitrary days. Meanwhile, in the example illustrated in FIG. 28, the degree of vitality is displayed as a health index. In the vitality degree web page 750, a user can perform selection for checking a vitality degree bar graph 755 with respect to one category or can compare the vitality degree bar graphs 755 in parallel with respect to two or more categories by using the vitality degree selection box 760. For example, the user may set only a bar graph for sleep to be in an operation state in order to check whether the overall grade of sleep has been improved compared to the previous month, or may compare the grade of sleep with the grade of an activity level corresponding thereto and evaluates the grades by simultaneously displaying the sleep and the activity level and may check whether there is some correlation between the days. The grade of nutrition and the grade of the degree of vitality may be displayed for a predetermined time interval so that it is checked whether there is some correlation between a daily dietary habit, a dietary habit during the interval, and the degree of vitality. FIG. 28 illustrates comparison between sleep and an activity level during a week from June 8 to June 14 using bar graphs, as an example for description. In addition, the vitality degree web page 750 also includes a tracing calculator 765 that displays access information, such as the sum of days in which a user has logged on and used the health manager, the proportion of days in which the user has used the health manager since admission, and the proportion of hours for which the user has used the sensor device in order to collect data, and statistics.

An example of the web page 550 serving as a starting point of the health manager illustrated in FIG. 22 includes summaries 556a to 556f of a plurality of categories, selectable by a user, which correspond to the categories of the health index 555 as the degree of health. Each of the summaries 556a to 556f of the respective categories presents a subset of data which is selected in advance with respect to the corresponding category and is filtered. The summary 556a of the category of nutrition indicates a daily target value and an actual value of a caloric intake. The summary 556b of the category of activity level indicates a daily target value and an actual value of the amount of calories burned. The summary 556c of the category of mental concentration indicates a target value and an actual value of the depth of mental concentration. The summary 556d of the category of sleep indicates a target sleep time, an actual sleep time, and the grade of the quality of sleep. The summary 556e of the category of daily activity displays a target point and an actual point based on a ratio of completed activity to a recommended healthy daily task (daily activity). The summary 556f of the category of the degree of vitality indicates a target grade and an actual grade of the degree of vitality of the day.

In addition, the web page 550 may also include a hyperlink (not shown) to a news story, comments (not shown) to a user based on a tendency such as malnutrition which is checked by the first examination, and a signal (not shown). The web page may also include a daily task portion 557 that provides information to a user every day. As comments of the daily task portion 557, for example, a water intake required every day, advice for specific means for enabling the intake of water, and the like can be displayed. In addition, the web page 550 may include a problem solution section 558 that actively evaluates a user's results in each category of the health index 555 and presents advice for improvement. For example, when a user's sleep level is “low” by a system and it is suggested that the user has insomnia, the problem solution section 558 can advise a method for improving sleep. In addition, the problem solution section 558 may include the user's question regarding an improvement in results. In addition, the web page 550 may include a daily data section 559 that starts up an input dialogue box. The user can easily input various pieces of data required by the health manager, using the input dialogue box. As known in the art, the input of data can be selectively performed between the input in a list presented in advance and the input in a general free text format. In addition, the web page 550 may include a body condition section 561 that gives information regarding life symptoms such as the height and weight of a user, a body measurement value, a BMI, a heart rate, blood pressure, or any physiological parameter.

What is claimed is:

1. A biological information measuring apparatus comprising:
a light emitting unit that emits light to a test subject;
a light receiving unit that receives light which is reflected from the test subject; and
a controller that determines whether at least one of the light receiving unit and the light emitting unit is brought into an intermittent operation.

2. The biological information measuring apparatus according to claim 1, further comprising:
a display unit that gives notice of information,
wherein the controller notifies the display unit that switching from a successive operation to the intermittent operation has been performed.

3. The biological information measuring apparatus according to claim 1, wherein the controller sets at least one of a stop time and a measurement time of the intermittent operation.

4. The biological information measuring apparatus according to claim 3, further comprising:
a secondary battery that accumulates power,
wherein the controller sets at least one of the stop time and the measurement time on the basis of at least one of a charging amount, remaining amount, and consumption of the secondary battery.

5. The biological information measuring apparatus according to claim 4, wherein the controller sets at least one of the stop time and the measurement time on the basis of a use time which is input by the test subject.

6. The biological information measuring apparatus according to claim 3, wherein the controller sets at least one of the stop time and the measurement time on the basis of biological information on the test subject which was measured in the past.

7. The biological information measuring apparatus according to claim 1, further comprising a plurality of the light emitting units.

8. The biological information measuring apparatus according to claim 2, wherein in a state of being set to the intermittent operation, the controller notifies the display unit of contents of the set intermittent operation.

9. The biological information measuring apparatus according to claim 2, wherein in a state of being set to the intermittent operation, the controller notifies the display unit of being in the measurement time.

10. The biological information measuring apparatus according to claim 2, wherein the controller notifies the display unit of an operable time.

11. The biological information measuring apparatus according to claim 1, further comprising an acceleration sensor.

12. The biological information measuring apparatus according to claim 11, wherein the controller sets an intermittent operation when the test subject is in a sleep state.

13. The biological information measuring apparatus according to claim 11, wherein the controller sets an intermittent operation on the basis of the test subject's lifestyle which is estimated by biological information of the test subject which was measured in the past.
